SEQUENCE LISTING (2 sequences)

SEQ ID NO: 1 - protein, 274 aa, Humicola lanuginosa
  VARIANT (1)..(274)
  mat_peptide (6)..(274)
SPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVRRRDIVKIEGIDATGGNNQPNIPDIPAHLWYFGLIGTCL

SEQ ID NO: 2 - protein, 269 aa, Humicola lanuginosa [sequence omitted]

(19) United States
(12) Patent Application Publication
(10) Pub. No.: US 2011/0008423 A1
(43) Pub. Date: Jan. 13, 2011

(54) PHARMACEUTICAL COMPOSITIONS COMPRISING GRANULES OF PURIFIED MICROBIAL LIPASE AND METHODS FOR PREVENTING OR TREATING DIGESTIVE DISORDERS

(75) Inventors: Florian Unger, Hannover (DE); George Shlieout, Sehnde (DE); Andreas Koerner, Springe (DE)

Correspondence Address: MAYER BROWN LLP, P.O. BOX 2828, CHICAGO, IL, 60690, US

(73) Assignee: ABBOTT PRODUCTS GMBH, Hannover (DE)

(21) Appl. No.: 12/811,410

(22) PCT Filed: Jan. 2, 2009
(86) PCT No.: PCT/EP09/50010

§ 371 Date: Sep. 27, 2010

Related U.S. Application Data
(60) Provisional application No. 61/018,834, filed on Jan. 3, 2008.

(30) Foreign Application Priority Data
Jan. 3, 2008 (EP) 08150018.3

(51) Int. Cl. A61K 38/46; A61K 9/16; A61K 9/48; A61K 9/20; A61P 1/18; A61P 3/10; A61P 5/48; B05D 5/00; B05D 7/00

(52) U.S. Cl. 424/452; 424/491; 424/94.6; 424/465; 427/2.14

(57) ABSTRACT

The present invention relates to pharmaceutical compositions comprising granules containing at least one recombinantly produced purified microbial lipase, the use of said pharmaceutical compositions for the manufacture of a medicament for the prevention or treatment of certain diseases or disorders like pancreatic endocrine insufficiency, and a process for the manufacture of said pharmaceutical compositions.

### CROSS-REFERENCE TO RELATED APPLICATIONS

This application is the National Stage of International Application No. PCT/EP2009/050010, filed Jan. 2, 2009, which claims the benefit of U.S. Provisional Application No. 60/018,834, filed Jan. 3, 2008 and EP 08150018.3, filed Jan. 3, 2008.

### FIELD

Described herein are pharmaceutical compositions comprising granules containing recombinantly produced purified microbial lipase, the use of said pharmaceutical compositions for the prevention or treatment of diseases and disorders, e.g., digestive disorders, a method of preventing or treating diseases and disorders by administering said pharmaceutical compositions to a mammal in need thereof, in particular a human, a process for the manufacture of said pharmaceutical compositions and pharmaceutical compositions prepared by said process.

### BACKGROUND

Enzymes, including lipases, are known for their use in various industrial applications, e.g., detergents and food industries. General reasons for formulating industrial enzymes into particles, such as enzyme granules or enzyme pellets, include, but are not limited to, protecting the enzymes from the surrounding potentially hostile environment until the active compound is to be released. A further reason relates to the reduction of potentially harmful dust, which may be generated from the enzymes upon handling.

U.S. Pat. No. 4,689,297 discloses a method for the preparation of dust-free enzyme -containing particles for use with laundry detergents. The enzyme containing particles are produced by coating hydratable core particles with the enzyme.

WO 91/06638 discloses a procedure for making dry and dust-free enzyme granules from a fermentation broth containing the enzyme for use in detergent and food applications. A fermentation broth is usually the media from which an enzyme produced by microbial processes is obtained. In addition to the particular enzyme, the fermentation broth usually contains a large number of by-products, e.g., oligosaccharides and polysaccharides.

More sophisticated formulations are usually applied to enzymes for pharmaceutical use. Several commercial medicaments in the form of pancreatic enzyme supplements are currently known for the treatment of diseases or disorders caused by digestive enzyme deficiency in mammals, such as humans. Active ingredients of these products, in particular digestive enzymes, namely amylase, lipase, and protease, are normally produced in the pancreas and excreted to the upper part of the small intestine. The enzymes used in such medicaments are often extracts from mammalian pancreatic glands, typically bovine or porcine pancreas.

European patent EP 0 583 726 B1 teaches an extrusion process for the production of pancreatin containing micropellets and micropellets obtainable by such process.

In WO 2007/02026012, pancreatin micropellet cores suitable for enteric coating are described which are produced by an extrusion process.

U.S. Pat. No. 4,079,125 discloses a process for preparing digestive enzyme compositions which may, inter alia, contain lipases. The compositions may comprise nonpareil seeds.

WO 93/07263 discloses a granular enzyme composition for use with detergents and having, inter alia, reduced tendencies to form dust and leave residue. The granular composition comprises a core, an enzyme layer and an outer coating layer.

Alternative methods for preparations comprising pancreatic enzymes formulations for pharmaceutical use are, e.g., disclosed in U.S. Pat. No. 4,447,412.

Enzymes or enzyme mixtures derived from microbial processes are also known, e.g., the product Nortase® which contains a lipase derived from Rhizopus oryzae, a protease derived from Aspergillus oryzae, and an amylase derived from Aspergillus oryzae.

The pharmaceutical use of certain microbial lipases is described in WO 2006/136159 A2 together with processes for their production and purification.

As the registration process for medicaments is very strict and safety data must be provided for all active ingredients thereof as well as for any by-products, such as degradation products, it is therefore preferred to produce medicaments with a high purity, with a high content of active ingredient and with the lowest possible content of by-products, e.g., by-products from degradation of the active ingredient. It has now been found that purified lipases, in particular purified recombinantly produced microbial lipases, need to be processed with particular care when manufactured into pharmaceutically administrable forms, e.g., granules for pharmaceutical use. For example, processing purified lipases, in particular recombinantly produced purified microbial lipases, by conventional extrusion techniques may result in the formation of peptidic impurities and thus in the loss of protein purity in the resulting purified microbial lipases. Such peptidic impurities may, e.g., result from degradation of the recombinantly produced purified microbial lipases themselves, e.g., due to mechanical stress during an extrusion process. The term “peptidic impurities” as used herein refers to the total degradation products of the recombinantly produced purified microbial lipases itself and further comprises all other peptidic and/or protein-derived by-products in a particular sample or product.

Thus, it is surprising, that a manufacturing process as described herein, wherein a solution comprising recombinantly produced purified microbial lipase is coated onto suitable pharmaceutically acceptable core particles, results in finished pharmaceutical compositions comprising granules of a particularly high protein purity of the recombinantly produced purified microbial lipase.

### SUMMARY

Described herein are pharmaceutical compositions comprising granules containing recombinantly produced purified microbial lipase, the use of said pharmaceutical compositions for the prevention or treatment of diseases and disorders, e.g., digestive disorders, a method of preventing or treating diseases and disorders by administering said pharmaceutical compositions to a mammal in need thereof, in particular a human, a process for the manufacture of said pharmaceutical compositions and pharmaceutical compositions prepared by said process.

Other embodiments, objects, features and advantages will be set forth in the detailed description of the embodiments that follows, and in part will be apparent from the description, or may be learned by practice, of the claimed invention. These objects and advantages will be realized and attained by the composition, methods and processes described and claimed herein. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### DETAILED DESCRIPTION

While the present disclosure is capable of being embodied in various forms, the description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

One embodiment described herein is a medicament comprising at least one recombinantly produced purified microbial lipase having a high content and protein purity, and with the lowest possible content of by-products. Accordingly, in another embodiment, the pharmaceutical composition comprises granules, said granules comprising:

a) a pharmaceutically acceptable core particle and

b) at least one coating layer coated on the core particle, said coating layer

comprising at least one recombinantly produced purified microbial lipase,

wherein said recombinantly produced purified microbial lipase has a protein purity of at least 90 area-% (w/w) and a protein content of at least 60% (w/w).

As used herein, a “granule” (or granules) is usually obtained as a particle of spherical or nearly spherical shape, the shape being mainly due to the related manufacturing process. In one embodiment, the size of the granules may vary across a broad range, but usually is a diameter of at least 100 μm, and in another embodiment at least 200 μm is used, in particular where the granules are for pharmaceutical use. Illustratively, granules for pharmaceutical use may be in a diameter of 200 to 4,000 μm, 300 to 3,000 μm, and of 400 to 2,000 μm.

In another embodiment, the pharmaceutical compositions described herein may be used in the prevention or treatment of digestive disorders, pancreatic exocrine insufficiency, pancreatitis, cystic fibrosis, diabetes type I and/or diabetes type II.

In one embodiment, the “core particles” of the pharmaceutical compositions as described herein can be pharmaceutically inactive and function as carriers for the active pharmaceutical ingredient of a pharmaceutical composition, viz. the recombinantly produced purified microbial lipase. Any type of pharmaceutically acceptable core particles known in the art for such purpose may be used, e.g., so called “non-pareil seeds” which are also sometimes referred to as “neutral pellets” or “starter pellets.” The core particles may consist of any pharmaceutically acceptable organic or inorganic material which is suitable for use to manufacture the granules as described herein, or of mixtures of such materials. A suitable inorganic material for the core particles is, e.g., silicon dioxide, in particular, coarse grade silicon dioxide. Suitable organic materials for the core particles are, e.g., cellulose, in particular, microcrystalline cellulose (“MCC”), starch and/or carbohydrates, such as sucrose or lactose. Organic materials, in particular cellulose, are preferred for the core particles. In one embodiment, the organic material is MCC. Typically, core particles of spherical or nearly spherical shape and of varying sizes are used. For pharmaceutical use, core particles of a diameter of at least 50 μm are usually used. Other embodiments include a diameter of 50 to 2,000 μm, 150 to 1,500 μm and 200 to 700 μm.

The granules of the pharmaceutical compositions as described herein also comprise at least one “coating layer.” The coating layer or layers comprises or comprise at least one recombinantly produced purified microbial lipase but may also comprise two or more of said lipases (the “recombinantly produced purified microbial lipase coating layer”). In one embodiment, there is one recombinantly produced purified microbial lipase per coating layer. Furthermore, in a further embodiment, the coating layer(s) or other elements of the pharmaceutical compositions described herein may optionally comprise enzyme stabilizing agents and/or binding agents as described below. In yet a further embodiment, the coating layer(s) or other elements of the pharmaceutical compositions described herein may optionally comprise additional conventional pharmaceutical auxiliaries and/or excipients as described below. Conventional coating materials may be used for the coating layer. The thickness of the coating layer may vary across a broad range and can be, e.g., 50 to 4,000 μm, 100 to 3,000 μm and 200 to 2,000 μm. In one embodiment, the coating layers are applied to the core particles by common coating techniques and may be applied in several layers, e.g., in two, three, four, five or more layers, over each other, as is known in the art. In another embodiment, there is one recombinantly produced purified microbial lipase coating layer.

The granules of the pharmaceutical compositions as described herein may further comprise one or more (i.e., two, three, four, five, six, seven, eight, nine, ten, or more) additional coating layers beside the “recombinantly produced purified microbial lipase coating layer.” In one embodiment, two or more recombinantly produced purified microbial lipase coating layers are comprised in the granule, the granule may optionally comprise one or more additional coating layers, e.g., for separating the recombinantly produced purified microbial lipase coating layer(s) from the surface of the core particle and/or from other recombinantly produced purified microbial lipase coating layers (“separating layer(s)”) or for providing a top coat applied on the surface of the recombinantly produced purified microbial lipase coating layer to protect the same from direct contact with the surrounding environment (“top coat layer(s)”). In another embodiment, the top coat layer comprises or consists of a functional (e.g., an enteric coating) coating. In a further embodiment, the top coat layer comprises or consists of a non-functional coating. In yet a further embodiment in which two or more coating layers are comprised in the granule of the pharmaceutical compositions as described herein, the two or more coating layers may be applied i) in direct contact to each other or ii) may be separated from each other by the application of one or more additional coating layers (i.e., separating layers). There are different ways to manufacture a granule containing more than one coating layer. In one embodiment, the granules are coated stepwise, i.e., add a first coating layer to the granule and then add a second coating layer to the granule. It might be necessary to dry the coated granules after each coating step. In an embodiment where more then two coating layers are needed, the additional coating layers are also added stepwise in the same or similar manner.

Additional coating layers and methods known in the art may be used, e.g., as described in documents DK 2002 00473, DK 2001 01930, WO 89/08694, WO 89/08695, and/or WO 00/01793. Other examples of coating materials may be found in U.S. Pat. No. 4,106,991, EP 170360, EP 304332, EP 304331, EP 458849, EP 458845, WO 97/39116, WO 92/12645 A, WO 89/08695, WO 89/08694, WO 87/07292, WO 91/06638, WO 92/13030, WO 93/07260, WO 93/07263, WO 96/38527, WO 96/16151, WO 97/23605, WO 01/25412, WO 02/20746, WO 02/28369, U.S. Pat. No. 5,879,920, U.S. Pat. No. 5,324,649, U.S. Pat. No. 4,689,297, U.S. Pat. No. 6,348,442, EP 206417, EP 193829, DE 4344215, DE 4322229 A, DE 263790, JP 61162185 A and/or JP 58179492, the disclosure of all of the aforementioned cited documents is incorporated herein by reference.

Suitable “enzyme stabilizing agents” for use with the coating layer(s) or with other elements of the pharmaceutical compositions described herein may, e.g., be non-reducing agents, such as non-reducing carbohydrates. In one embodiment, the enzyme stabilizing agents are selected from the group consisting of: sucrose, trehalose, and maltitol. The enzyme stabilizing agents are used in an amount of 0-100% (w/w) per weight of purified lipase. In another embodiment, the enzyme stabilizing agents are used in an amount of 10-100% (w/w). In one embodiment, the enzyme stabilizing agents is used with the coating layer(s).

Suitable “binding agents” for use with the coating layers or with other elements of the pharmaceutical compositions described herein may, e.g., be agents with a high melting point or no melting point at all and optionally of a non-waxy nature. For example, cellulose derivatives may be used as suitable binding agents, such as hydroxypropylmethylcellulose (“hypromellose”), hydroxypropylcellulose, methylcellulose or carboxymethylcellulose. Furthermore, suitable binding agents may be selected from the group consisting of: polyvinylpyrrolidon (“PVP”); dextrine; and polyvinylalcohol. The binding agents are used in an amount of 0-20% (w/w) per weight of recombinantly produced purified microbial lipase. In another embodiment, the binding agent is in an amount of 2.5-10% (w/w). In a further embodiment, the binding agents is used with the coating layer(s).

The “recombinantly produced purified microbial lipase” as described herein for the use with the granules of the pharmaceutical composition according to the present disclosure, has a protein purity of at least 90 area-%, 91 area-%, 92 area-%, 93 area-%, 94 area-%, 95 area-%, 96 area-%, 97 area-%, 98 area-%, 99 area-%, preferably of at least 99.1 area-%, 99.2 area-%, 99.3 area-%, 99.4 area-%, 99.5 area-%, 99.6 area-%, 99.7 area-%, 99.8 area-% or 99.9 area-%. As described herein, the term “protein purity” is to be understood as the percentage of recombinantly produced purified microbial lipase protein mass based on the total protein mass present in a specific sample or product, e.g., a specific sample of a recombinantly produced purified microbial lipase. In one embodiment, the protein purity of the recombinantly produced purified microbial lipase as described herein can be measured by a chromatographic method.

The chromatographic peaks obtained are quantified by the area-% method and the area-% of the lipase peaks are expressed as percentage of the total area of all detected peaks. In one embodiment, the protein purity is measured by Reversed Phase-High Performance Liquid Chromatography (“RP-HPLC”) , and in another embodiment by gradient RP-HPLC. Gradient RP-HPLC is performed with a suitable solvent, e.g., consisting of acetonitrile, water and trifluoro acetic acid (“TFA”). The separation is performed on a suitable HPLC column, such as a YMC Protein RP, S-5 μm column, 125×3 mm I.D. (YMC Europe GmbH, Schermbeck, Germany) by running a suitable gradient, such as a gradient from 0 to 90% acetonitrile/TFA 0.05%, within a suitable time, such as within 50 min, at a suitable flow rate, such as at a flow rate of 1.0 ml/min. The detection is to be performed at a suitable wavelength, such as a wavelength of 214 nm. The sample to be examined is to be dissolved in a suitable solvent, such as an aqueous solution of sodium chloride 2% (w/w). The column is operated at a suitable temperature, such as 40° C. In one embodiment, when used as a starting material to produce the granules of the pharmaceutical composition according to the present disclosure, the recombinantly produced purified microbial lipase may have a protein purity of at least 90 area-% and as high as 99.9 area-%. This protein purity may decrease during the manufacturing process and the degree of any decrease will typically depend on the manufacturing process applied. Due to the very gentle conditions which occur during the process as described herein to manufacture the granules comprising recombinantly produced purified microbial lipase, the losses in protein purity during said formulation process are low, e.g., below 0.5%. For example, if a recombinantly produced purified microbial lipase of a 99.9 area-% protein purity is used as a starting material in a process as described herein to manufacture the granules comprising recombinantly produced purified microbial lipase, the protein purity of the recombinantly produced purified microbial lipase in the resulting granule may typically be as high as 99.6 area-%.

In one embodiment, the specific activity of the recombinantly produced purified microbial lipase as described herein is at least 80% of its maximum specific activity (as described below). In another embodiment, the specific activity of the recombinantly produced purified microbial lipase as described herein is at least 81, 82, 83, 84, 85, 86, 87, 88, 89, 90, 91, 92, 93, 94, 95, 96, or 97%, respectively, of its maximum specific activity. The “specific activity” of an enzyme (e.g., a lipase) is the enzyme activity (e.g., the lipolytic activity) based on the total weight of enzyme protein. In yet another embodiment, the recombinantly produced purified microbial lipase is used in a solid form, e.g., in the form of a powder, crystals, microcrystals or the like.

The term “total protein mass” is to be understood as the sum of recombinantly produced purified microbial lipase protein and peptidic and/or protein-derived impurities, including degradation products of the recombinantly produced purified microbial lipase. In one embodiment, the total protein mass does not comprise any added proteins such as other enzymes (non-lipases), peptidic excipients, and/or protein-derived excipients. The desired protein purity of a recombinantly produced purified microbial lipase can be achieved as described in more detail below. The recombinantly produced purified microbial lipase as described herein has a protein content of at least 60% (w/w), 65%, 70%, 75%, 76%, 77%, 78%, 79%, 80%, 81%, 82%, 83%, 84%, 85%, 86%, 87%, 88%, 89%, 90%, 91%, 92%, 92%, 93%, 94% or 95%.

As described herein, the term “protein content” is to be understood as the percentage of lipase protein mass based on the total mass of lipase preparation, the lipase preparation comprising lipase protein and non-peptidic constituents like, e.g., oligosaccharides, polysaccharides, salts, residual water, and the like. In one embodiment, the raw lipase preparation for obtaining recombinantly produced purified microbial lipase is obtained from the fermentation broth in a known manner, with a subsequent further purification and/or drying step carried out on the lipase preparation where desired or needed. If a recombinantly produced purified microbial lipase of a protein content of 60% (w/w) or higher is desired, the lipase preparation is usually dried after it has been recovered from the fermentation broth. In one embodiment, the lipase preparation to be dried can be a liquid lipase concentrate. Drying is usually carried out as spray-drying or freeze drying. In one embodiment, drying is spray-drying. For example, in one embodiment, when used as a starting material to produce the granules of the pharmaceutical composition, the recombinantly produced purified microbial lipase may have a protein content of at least 60%, 65%, 70%, 75%, 76%, 77%, 78%, 79% (w/w in each case), or at least 80% (w/w). In another embodiment, the recombinantly produced purified microbial lipase has a protein content of at least 80% (w/w). Where pharmaceutical compositions are concerned, any determined protein content therein will typically be lower than the protein content present in the recombinantly produced purified microbial lipase used as starting material to produce the granules of the pharmaceutical composition. This is due to the presence of additional substances like pharmaceutical auxiliaries and/or excipients in the granules of the pharmaceutical compositions.

The protein content can be determined by the “external standard method,” i.e., relative to a solution of a “lipase protein reference standard” (“LRS”) with a defined protein content based on the amino acid composition of a particular lipase which is determined independently (for details see Example 6). According to the “Analytical Procedures and Methods Validation” (guidance provided by the US Food and Drug Administration, August 2000) reference standards from the United States Pharmacopeia (USP)/National Formulary (NF) and other official sources do not require further characterization. A reference standard that is not obtained from an official source should be of the highest purity that can be obtained by reasonable effort, and it should be thoroughly characterized to ensure its identity, strength, quality, purity, and potency. The qualitative and quantitative analytical procedures used to characterize a reference standard are expected to be different from, and more extensive than, those used to control the identity, strength, quality, purity, and potency of the drug substance or the drug products. However, for drug applications for new molecular entities, it is unlikely that an international or national standard will be available. The manufacturer should therefore establish an appropriately characterized in-house primary reference material. In-house working reference material(s) used in the testing of production lots should be calibrated against this primary reference material. In the field of enzymes, the reference standard is characterized by having the highest available purity (e.g., higher than 99.9%). Due to its very high purity, the specific activity of an enzyme reference standard usually represents the “maximum specific activity” (or the approximate maximum specific activity which may usually be equated with the maximum specific activity for practical purposes due to the usually very low deviations of the numerical values between the maximum specific activity and the approximate maximum specific activity) of this specific enzyme, when determined under applicable standard conditions for said specific enzyme.

The desired protein content of a purified lipase, in particular of a recombinantly produced microbial purified lipase, can be achieved as described in more detail below. In one embodiment, the recombinantly produced microbial purified lipase has a specific activity of at least 1,000,000 U/g. In one embodiment, the specific activity of a lipase can be determined as described in Example 8. The unit of the lipase activity “U/g” is to be understood as “units per gram of enzyme protein”. One unit (U) is defined as the enzymatic activity which hydrolyses 1 μequivalent of titratable fatty acid within one minute at a pH of 7.0 at 37° C. under certain conditions. The lipase activity (synonymously used expressions are “enzyme activity,” “enzymatic activity” and “lipolytic activity”) is to be understood as the moles converted per unit time as defined in Example 7.

For the purposes of the present disclosure, a “lipase” means a carboxylic ester hydrolase EC 3.1.1. -, which includes activities such as EC 3.1.1 3 triacylglycerol lipase, EC 3.1.1.4 phospholipase A1, EC 3.1.1.5 lysophospholipase, EC 3.1.1.26 galactolipase, EC 3.1.1.32 phospholipase A1, EC 3.1.1.73 feruloyl esterase. In another embodiment, the lipase is an EC 3.1.1.3 triacylglycerol lipase. The EC number refers to Enzyme Nomenclature 1992 from NC-IUBMB, Academic Press, San Diego, Calif., including supplements 1-5 published in Eur. J. 25 Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see, e.g., the World Wide Web at http://www.chem.qmw.ac.uk/iubmb/enzyme/index.html.

Lipases may be plant-derived or of animal, in particular mammal, fungal or bacterial origin. In another embodiment, said fungi or bacteria producing fungal or bacterial lipases are recombinant fungi or bacteria. For example, microbial lipases may be recovered from a fermentation broth and mammal lipases may be recovered from pancreas swine or bovine extracts by conventional procedures including, but not limited to, centrifugation, filtration, extraction, spray-drying, evaporation, or precipitation.

According to the present disclosure, any recombinantly produced microbial lipase suitable for pharmaceutical use may be used. In one embodiment, the lipase should be suitable to prevent or treat diseases and disorders, preferably digestive disorders, pancreatic exocrine insufficiency, pancreatitis, cystic fibrosis, diabetes type I and/or diabetes type II.

A recombinantly produced microbial lipase is an enzyme produced by the way of recombinant DNA-technology, the lipase being of microbial origin, i.e., obtained from fungi or bacteria. In one embodiment, suitable lipases are recombinantly produced microbial lipases that possess lipolytic activity, preferably, at relatively low pH. In another embodiment, the recombinantly produced microbial lipase may be an enzyme variant or a mutated enzyme being functionally equivalent or having structural features similar to a naturally occurring lipase. An enzyme variant or mutated enzyme is obtainable by alteration of the DNA sequence of the parent gene or its derivatives. The enzyme variant or mutated enzyme may be expressed and produced when the DNA nucleotide sequence encoding the respective enzyme is inserted into a suitable vector in a suitable host organism. The host organism does not necessarily have to be identical to the organism from which the parent gene originated. The methods for introducing mutations into genes are well known in the art, see, e.g., patent application EP 0 407 225.

In one embodiment, recombinantly produced microbial lipases are lipases derived from fungi, e.g., from Humicola, Rhizomucor, Rhizopus, Geotrichum or Candida species, in particular Humicola lanuginosa (Thermomyces lanuginosa), Rhizomucor miehei, Rhizopus javanicus, Rhizopus arrhizus, Rhizopus oryzae, Rhizopus delamar, Candida cylindracea, Candida rugosa or Geotrichum candidum; or may be derived from bacteria, e.g., from Pseudomonas, Burkholderia or Bacillus species, in particular Burkholderia cepacia. Another embodiment are lipases derived from a strain of Humicola lanuginosa (Thermomyces lanuginosa) or Rhizomucor miehei. A further embodiment are lipases derived from a strain of Humicola lanuginosa (Thermomyces lanuginosa).

Lipases of microbial origin and their production by, e.g., recombinant technology are described in, e.g. EP Publication Nos. 0600868, 0238023, 0305216, 0828509, 0550450, 1261368, 0973878 and 0592478, which publications are hereby incorporated by reference. EP publication No. 0600868 (U.S. Pat. No. 5,614,189) describes, inter alia, the use of a lipase derived from Humicula lanuginosa in pancreatic enzyme replacement therapy. Said lipase is from Humicula lanuginosa DSM 4109 and has the amino acid sequence of amino acids 1-269 of SEQ ID NO:2.

In one embodiment, a lipase, derived from Humicola lanuginosa which comprises an amino acid sequence having at least 80% identity to the amino acids 1-269 of SEQ ID NO: 2, may be used to prepare recombinantly produced purified microbial lipase. In a further embodiment, a lipase, derived from Humicola lanuginosa which has at least 80% identity to the amino acids 1-269 of SEQ ID NO: 2, may be used to prepare recombinantly produced purified microbial lipase. In yet a further embodiment, a lipase, derived from Humicola lanuginose having SEQ ID NO: 2, may be used to prepare recombinantly produced purified microbial lipase.

In one embodiment, the lipases for use as a medicament disclosed in WO 2006/136159 and/or in International Patent Application WO 2008/079685 (PCT/US07/87168), preferably as set out in the respective claims, may be used according to the present disclosure. The disclosures of the documents WO 2006/136159 and WO 2008/079685 are both incorporated herein by reference in their entireties.

Accordingly, the recombinantly produced purified microbial lipase to be used in one embodiment in the context of the present disclosure:
(a) has at least 50%, 60%, 70%, 80% or 90% identity to the sequence of amino acids 1 to 269 of SEQ ID NO: 2;
(b) has lipase activity; and
(c) optionally, as compared to the sequence of amino acids 1-269 of SEQ ID NO: 2, comprises:
   (i) substitutions T231R and N233R; or
   (ii) substitutions N33Q, T231R, and N233R; or
   (ii) substitutions N33Q, T231R, and N233R; as well as at least one additional substitution selected from the following:
E1*,D,N; Q4H,P,R; D5E; N8L,Q; Q9H; F10L; N11C,D,H,L,P,Q,R,S; G23E; N26A,H,I; D27I,N,Q,R,S,V; P29T; A30T,V; T37K,M; G38A,D,F,H,I,K,L,M,N,P,Q,S,T,W,Y; N39H,S; E43K; K46M; A49T; L52I,R; E56K,Q,R,S; D57G,N; V60E,S; G61R; V63R; A68V; L69I; N71I,S; N73Q,Y; I76T; R84E; I86F,L; E87A,H,K,R; I90L,V; G91A,C,E,F,K,L,M,N,S,T,V,W,Y; L93*,F; N94*,K,Q,R,S; F95*; D96*,E,G,N,R,S,W,Y; L97M,Q; K98,I,T; E99D; N101Q; D102E,G,Y; R108M; G109A; D111A,E,N,S; G112A; T114I; S115L; W117C,D,E,F,G,H,I,K,L,P,S,T,V,Y; D122E,N; Q126L; V128A; D130H; H135D; P136H; Y138F; V141E,L; A150V; V154F,I,L; A155V; G156R; G161A,E; N162G,S,T; G163A,C,D,E,H,I,K,L,M,N,P,Q,R,S,T,V,W,Y; D167E; V168M; V176A,D,F,G,H,I,K,M,N,Q,T,W; G177A; R179T; L185M; G190C,D; N200Q,S; R205I; L206F; E210D,R,V,Y; S216P; E219D; G225P; T226N; L227F,G; P229R; E239D; G240L; D242E; T244S; G246A; Q249R; N251Q,S; D254A,G,I,K,L,M,N,R,Q,S,Y; I255A,F; P256A,F,G,H,I,L,M,N,Q,S,T,V,W,Y; and L269F,H.

A recombinantly produced purified microbial lipase to be used in an embodiment in the context of the present disclosure has at least 90% identity to amino acids 1-269 of SEQ ID NO: 1. SEQ ID NO: 1 differs from amino acids 1-269 of SEQ ID NO: 2 by the double-substitution T231R+N233R. The expression “the double substitution T231R+N233R” in SEQ ID NO: 1 refers to the fact that a variant SEQ ID NO: 1 is a variant of SEQ ID NO: 2, in which the threonine (Thr, or T) residue in position 231 and the asparagine (Asn, or N) residue in position 233 have each been substituted by an arginine residue (Arg, or R). The term “position” refers to the positive amino acid residue numbers in SEQ ID NO: 1 of the sequence listing. These two substitutions are not conservative, as defined below (since they replace two basic amino acids with two polar amino acids).

In additional embodiments, the recombinantly produced purified microbial lipase is at least 81%, 82%, 83%, 84%, 85%, 86%, 87%, 88%, 89%, 90%, 91%, 92%, 93%, 94%, 95%, 96%, 97%, 98%, or at least 99% identical to SEQ ID NO: 1.

In one embodiment, the recombinantly produced purified microbial lipase
a) comprises amino acids 1-269 of SEQ ID NO: 1, or
b) is a variant of amino acids 1-269 of SEQ ID NO: 1, wherein the variant differs from amino acids 1-269 of SEQ ID NO: 1 by no more than twenty-five amino acids, and wherein:
   (i) the variant comprises at least one conservative substitution and/or insertion of one or more amino acids as compared to amino acids 1-269 of SEQ ID NO: 1; and/or
   (ii) the variant comprises at least one small deletion as compared to amino acids 1-269 of SEQ ID NO: 1; and/or
   (iii) the variant comprises at least one small N- or C-terminal extension as compared to amino acids 1-269 of SEQ ID NO: 1; and/or
   (iv) the variant is a fragment of the lipase having amino acids 1-269 of SEQ ID NO: 1.

Lipases comprising conservative substitutions, insertions, deletions, N-terminal extensions, and/or C-terminal extensions, as well as lipase fragments, as compared to the sequence of amino acids 1-269 of SEQ ID NO: 1, can be prepared from this molecule by any method known in the art, such as site-directed mutagenesis, random mutagenesis, consensus derivation processes (EP 897985), and gene shuffling (WO 95/22625, WO 96/00343). Such lipases may also be hybrids, or chimeric enzymes.

The variant lipase to be used in the embodiments of the disclosure of course has lipase activity. In one embodiment, the specific activity of the variant lipase is at least 50% of the specific activity of the lipase having amino acids 1-269 of SEQ ID NO: 1. In additional embodiments, the specific activity of the variant lipase is at least 60, 70, 75, 80, 85, 90, or at least 95% of the specific activity of the lipase having amino acids 1-269 of SEQ ID NO: 1, whereby the specific activity may be measured using the lipase assay of Example 8 as described herein, or using any of the lipase assays as set out in Example 1 of WO 2006/136159. In one embodiment, the specific activity is measured in U/mg enzyme protein using the LU-assay of Example 1 of WO 2006/136159, and determining enzyme protein content by amino acid analysis as described in Example 5 of WO 2006/136159.

The amino acid changes allowed for in the lipase variant of SEQ ID NO:1 are of a minor nature, i.e., conservative amino acid substitutions or insertions that do not significantly affect the folding and/or activity of the protein, preferably a small number of such substitutions or insertions; small deletions; small amino- or carboxyl-terminal extensions, such as an amino-terminal methionine residue; a small linker peptide; or a small extension that facilitates purification by changing net charge or another function, such as a poly-histidine tract, an antigenic epitope, or a binding domain.

In the above context, the term “small” independently designates a number of up to 25 amino acid residues. In one embodiment, the term “small” independently designates up to 24, 23, 22, 21, or up to 20 amino acid residues. In additional embodiments, the term “small” independently designates up to 19, 18, 17, 16, 15, 14, 13, 12, 11, or up to 10 amino acid residues. In further embodiments, the term “small” independently designates up to 9, 8, 7, 6, 5, 4, 3, 2, or up to 1 amino acid residue. In yet further embodiments, the term “small” independently designates up to 40, 39, 38, 37, 36, 35, 34, 33, 32, 31, 30, 29, 28, 27, 26, or up to 25 amino acid residues.

In one embodiment, the recombinantly produced purified microbial lipase has an amino acid sequence which differs by no more than 25, 24, 23, 22, 21, 20, 19, 18, 17, 16, 15, 14, 13, 12, or no more than 11 amino acids from amino acids 1-269 of SEQ ID NO: 1; or, it differs from amino acids 1-269 of SEQ ID NO: 1 by no more than 10, 9, 8, 7, 6, 5, 4, 3, 2, or by no more than 1 amino acid; in either case, preferably with the exception of the double substitution R231T+R233N in SEQ ID NO: 1 as defined above. In alternative embodiments, the lipase has an amino acid sequence which differs by no more than 40, 39, 38, 37, 36, 35, 34, 33, 32, 31, 30, 29, 28, 27, or no more than 26 amino acids from amino acids 1-269 of SEQ ID NO: 1, preferably, with the exception of the double substitution R231T+R233N in SEQ ID NO: 1, as defined above.

Examples of conservative substitutions are within the group of basic amino acids (arginine, lysine and histidine), acidic amino acids (glutamic acid and aspartic acid), polar amino acids (serine, threonine, glutamine and asparagine), hydrophobic amino acids (leucine, isoleucine, valine and alanine), aromatic amino acids (phenylalanine, tryptophan and tyrosine), and small amino acids (glycine, alanine, proline, serine, threonine, cysteine and methionine).

In the alternative, examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Another embodiment of a variant lipase which can be used in the context of the present disclosure is variant Asn33Gln (N33Q) of amino acids 1-269 of SEQ ID NO: 1. comprising a conservative substitution (exchange of one polar amino acid for another polar amino acid). This is a non-glycosylated variant which is as efficient as SEQ ID NO: 1 for the purposes of the present disclosure. Another embodiment relates to the use of this variant lipase as such, as well as to the correspondingly substituted variants of amino acids −5-269, −4-269, −3-269, and 2-269 of SEQ ID NO: 1.

In another embodiment, each of the substitutions in the variant lipase of the recombinantly produced purified microbial lipase is conservative.

Examples of variant lipases which can be used in the present disclosure comprise small N-terminal extensions are amino acids −5-269 (−5 to +269), −4-269 (−4 to +269), and −3-269 (−3 to +269) of SEQ ID NO: 1, viz. with the N-terminals of SPI . . . , PIR . . . , and IRR . . . , respectively (see Example 11).

One embodiment of a variant lipase which can be used in the present disclosure is the variant having the amino acid sequence of amino acids 2-269 (+2 to +269) of SEQ ID NO: 1, viz. with the N-terminus of VSQ (see Example 11) is which a fragment of amino acids 1-269 of SEQ ID NO: 1.

The lipases with the following amino acid sequences are embodiment of purified lipases to be used in the context of this disclosure: (i) amino acids +1 to +269 of SEQ ID NO: 1, (ii) amino acids −5 to +269 of SEQ ID NO: 1; (iii) amino acids −4 to +269 of SEQ ID NO: 1; (iv) amino acids −3 to +269 of SEQ ID NO: 1; (v) amino acids −2 to +269 of SEQ ID NO: 1; (vi) amino acids −1 to +269 of SEQ ID NO: 1, (vii) amino acids +2 to +269 of SEQ ID NO: 1, as well as (viii) any mixture of two or more of the lipases of (i)-(vii). In one embodiment, the lipase is selected from the lipases of (i), (ii), and any mixture of (i) and (ii). In one embodiment, mixtures of (i) and (ii) comprise at least 5% of the percentage being determined by N-terminal sequencing using the Edman method, as described in Example 11. In another embodiment, mixture of (i) and (ii) comprise at least 10%, 20%, 30%, 40%, 50%, 60%, 70%, 80%, 90%, or at least 95% of lipase (i), the percentages being determined by N-terminal sequencing using the Edman method, as described in Example 11. Other embodiment of mixtures include: (a) compositions comprising 35-75%, 40-70% and 45-65% of lipase (ii); (b) compositions comprising 20-60%, 25-55%, 30-50%, and 35-47% of lipase (i); (c) compositions comprising up to 30%, up to 25%, up to 20% and up to 16% of lipase (vii); and (d) any combination of (a), (b), and/or (c), such as a composition comprising 45-65% of lipase (ii), 35-47% of lipase (i), and up to 16% of lipase (vii).

In another embodiment, the recombinantly produced purified microbial lipase may also be a fragment of the lipase having amino acids 1-269 of SEQ ID NO: 1, whereby the fragment still has lipase activity. The term fragment is defined herein as a polypeptide having one or more amino acids deleted from the amino and/or carboxyl terminus of SEQ ID NO: 1, such as from the mature part thereof (amino acids 1-269 thereof). In one embodiment, a small number of amino acids has been deleted, small being defined as explained above. In a further embodiment, a fragment contains at least 244, 245, 246, 247, 248, 249, or at least 250 amino acid residues. In a yet further embodiment, a fragment contains at least 251, 252, 253, 254, 255, 256, 257, 258, 259, 260, 261, 262, 263, 264, 265, 266, 267, or at least 268 amino acid residues. In an alternative embodiment, a fragment contains at least 239, 240, 241, 242, or at least 243 amino acid residues.

In one embodiment, the lipase to be used as recombinantly produced purified microbial lipase is a variant of a parent lipase as disclosed in WO/2008/079685, which
(a) has at least 50% identity to the sequence of amino acids 1 to 269 of SEQ ID NO: 2;
(b) has lipase activity; and which
(c) as compared to the sequence of amino acids 1-269 of SEQ ID NO: 2, comprises substitutions N33Q, T231R, and N233R, as well as at least one additional substitution selected from the following:
E1*,D,N; Q4H,P,R; D5E; N8L,Q; Q9H; F10L; N11C,D,H,L,P,Q,R,S; G23E; N26A,H,I; D27I,N,Q,R,S,V; P29T; A30T,V; T37K,M; G38A,D,F,H,I,K,L,M,N,P,Q,S,T,W,Y; N39H,S; E43K; K46M; A49T; L52I,R; E56K,Q,R,S; D57G,N; V60E,S; G61R; V63R; A68V; L69I; N71I,S; N73Q,Y; I76T; R84E; I86F,L; E87A,H,K,R; I90L,V; G91A,C,E,F,K,L,M,N,S,T,V,W,Y; L93*,F; N94*,K,Q,R,S; F95*; D96*,E,G,N,R,S,W,Y; L97M,Q; K98I,T; E99D; N101Q; D102E,G,Y; R108M; G109A; D111A,E,N,S; G112A; T114I; S115L; W117C,D,E,F,G,H,I,K,L,P,S,T,V,Y; D122E,N; Q126L; V128A; D130H; H135D; P136H; Y138F; V141E,L; A150V; V154F,I,L; A155V; G156R; G161A,E; N162G,S,T; G163A,C,D,E,H,I,K,L,M,N,P,Q,R,S,T,V,W,Y; D167E; V168M; V176A,D,F,G,H,I,K,M,N,Q,T,W; G177A; R179T; L185M; G190C,D; N200Q,S; R205I; L206F; E210D,R,V,Y; S216P; E219D; G225P; T226N; L227F,G; P229R; E239D; G240L; D242E; T244S; G246A; Q249R; N251Q,S; D254A,G,I,K,L,M,N,R,Q,S,Y; I255A,F; P256A,F,G,H,I,L,M,N,Q,S,T,V,W,Y; and L269F,H.

In another embodiment, the lipase to be used as recombinantly produced purified microbial lipase is a variant of a parent lipase, which
(a) has at least 50% identity to the sequence of amino acids 1 to 269 of SEQ ID NO: 2;
(b) has lipase activity; and which
(c) as compared to the sequence of amino acids 1-269 of SEQ ID NO: 2, comprises a set of substitutions selected from the following:
D27R+N33Q+G91A+D96E+L97Q+D111A+T231R+N233R+P256T;
N33Q+E210D+T231R+N233R;
N33Q+D111A+T231R+N233R;
N33Q+G91T+T231R+N233R;
N33Q+E219D+T231R+N233R;
N33Q+W117L+T231R+N233R;
D27Q+N33Q+T231R+N233R;
N33Q+G91T+T231R+N233R;
D27S+N33Q+G91A+D96E+L97Q+D111A+S216P+T231R+N233R+P256T;
D27R+N33Q+G91N+N94R+D111A+T231R+N233R+P256T;
D27R+N33Q+G91T+N94S+D111A+S216P+L227G+T231R+N233R+P256T;
Q4R+N33Q+T231R+N233R;
N33Q+T231R+N233R+Q249R;
N33Q+D96W+T231R+N233R;
D27V+N33Q+V60S+D96W+T231R+N233R+Q249R;
D27V+N33Q+V60S+T231R+N233R+Q249R;
Q9H+N33Q+D102E+T231R+N233R;
N33Q+D111E+T231R+N233R;
N33Q+D122E+T231R+N233R;
D27R+N33Q+G91N+N94R+D111A+S216P+L227G+T231R+N233R+P256T;
N33Q+D167E+T231R+N233R;
N33Q+G91N+T231R+N233R;
N33Q+T231R+N233R+P256T;
D27R+N33Q+G91A+L93*+N94*+F95*+D96*+D111A+T231R+N233R+P256T;
N11R+N33Q+T231R+N233R;
N33Q+N39H+T231R+N233R;
N33Q+P229R+T231R+N233R;
D27R+N33Q+G91N+N94R+D111A+G163K+S216P+L227G+T231R+N233R+P256T;
N33Q+G91T+G163K+T231R+N233R;
D27R+N33Q+G91A+D96E+L97Q+D111A+S216P+L227G+T231R+N233R+P256T;
D27R+N33Q+G91A+D96E+L97Q+D111A+S216P+T231R+N233R+P256T;
N33Q+E87A+T231R+N233R;
N33Q+E56Q+T231R+N233R;
N33Q+E210V+T231R+N233R;
N33Q+E56K+T231R+N233R;
N33Q+T231R+N233R+D254G;
N33Q+D96S+T231R+N233R;
N33Q+D122N+T231R+N233R;
N26A+N33Q+T231R+N233R;
N33Q+N162T+T231R+N233R;
N33Q+A150V+N162G+T231R+N233R;
N33Q+I90L+G163L+T231R+N233R;
N33Q+T231R+N233R+G240L;
D27R+N33Q+G91A+D96E+D111A+T231R+N233R+D254G+P256T;
D27R+N33Q+G91A+N94S+D111A+T231R+N233R+P256T;
N33Q+N200S+T231R+N233R;
N33Q+N39S+T231R+N233R;
N33Q+E210R+T231R+N233R;
N33Q+N39H+T231R+N233R+D254R;
N33Q+T231R+N233R+D254R;
N33Q+N94R+T231R+N233R;
N33Q+D96R+T231R+N233R;
D27N+N33Q+T231R+N233R;
D27N+N33Q+E56R+T231R+N233R;
N33Q+L227F+T231R+N233R;
N33Q+N73Y+G225P+T231R+N233R;
N33Q+G225P+T231R+N233R;
N33Q+T231R+N233R+D254S;
N33Q+D96G+T231R+N233R;
N33Q+D96N+T231R+N233R+D254S;
N33Q+T231R+N233R+D254G;
N33Q+D130H+T231R+N233R;
N33Q+E87A+T231R+N233R;
N33Q+T231R+N233R+E239D;
N33Q+D111A+T231R+N233R+D254G;
N33Q+E210V+T231R+N233R+D254S;
N11R+N33Q+E210V+T231R+N233R+D254S;
N33Q+G91T+G163K+T231R+N233R+D254G;
N33Q+G91T+G163K+T231R+N233R+D254S;
N11R+N33Q+G91T+G163K+T231R+N233R+D254S;
Q4R+D27R+N33Q+G91T+N94S+D111A+S216P+L227G+T231R+N233R+P256T;
N33Q+G91T+N94S+D111A+V176I+T231R+N233R;
Q4R+D27R+N33Q+G91T+N94S+D111A+E210D+S216P+L227G+T231R+N233R+P256T;
Q4R+D27Q+N33Q+G91T+N94S+D111A+S216P+L227G+T231R+N233R+P256T;
N33Q+G91T+N94S+D111A+T231R+N233R+P256T;
N33Q+G177A+T231R+N233R;
N33Q+T231R+N233R+G246A;
D27N+N33Q+G91T+G163K+T231R+N233R+D254S;
D27Q+N33Q+G91T+G163K+E219D+T231R+N233R;
N33Q+G91T+E219D+T231R+N233R;
K98I+T231R+N233R+N251S;
N33Q+G163R+T231R+N233R;
N33Q+G163N+T231R+N233R;
N33Q+G163C+T231R+N233R;
N33Q+G163Q+T231R+N233R;
N33Q+G163E+T231R+N233R;
N33Q+G163H+T231R+N233R;
N33Q+G163I+T231R+N233R;
N33Q+G163P+T231R+N233R;
N33Q+G163D+T231R+N233R;
N33Q+G91K+T231R+N233R;
N33Q+G91M+T231R+N233R;
N33Q+G91F+T231R+N233R;
N33Q+G91S+T231R+N233R;
N33Q+G91W+T231R+N233R;
N33Q+G91Y+T231R+N233R;
N33Q+G163T+T231R+N233R;
N33Q+G163W+T231R+N233R;
N33Q+G163Y+T231R+N233R;
N33Q+G163V+T231R+N233R;
N33Q+G91C+T231R+N233R;
N33Q+G91Y+Q126L+T231R+N233R;
N33Q+G91M+G161E+T231R+N233R;
N33Q+V128A+T231R+N233R;
N33Q+G163V+L185M+T231R+N233R;
N33Q+G38A+T231R+N233R;
N33Q+G163A+T231R+N233R;
N33Q+G91T+N94S+D111A+T231R+N233R;
N33Q+G38A+G163A+T231R+N233R;
N33Q+G163M+T231R+N233R;
N33Q+G91V+T231R+N233R;
N33Q+D111A+T231R+N233R+Q249R;
N33Q+D111A+T231R+N233R+D254S;
D27R+N33Q+G91A+D96E+L97Q+D111A+T231R+N233R+D254S+P256T;
D27R+N33Q+G91A+D96E+L97Q+D111A+T231R+N233R+D254G+P256T;
N33Q+G91T+N94R+T231R+N233R+D254S;
N33Q+G91T+N94R+D111A+W117L+T231R+N233R;
N33Q+W117L+T231R+N233R+D254S;
N33Q+T231R+N233R+P256T;
N33Q+T231R+N233R+D242E;
N33Q+E87R+T231R+N233R;
N33Q+E56R+T231R+N233R;
N33Q+N162G+T231R+N233R;
N33Q+G91L+T231R+N233R;
N33Q+E87H+T231R+N233R;
N33Q+D96N+T231R+N233R+Q249R;
N33Q+G91T+N94R+T231R+N233R+D254S;
N33Q+L227F+T231R+N233R+D254S;
D27R+N33Q+G91T+D96E+L97Q+D111A+T231R+N233R+D254S+P256T;
N33Q+G163A+T231R+N233R;
D27R+N33Q+G91T+D96E+D111A+T231R+N233R+D254S+P256T;
N33Q+G91T+N94R+T231R+N233R;
N33Q+T231R+N233R+D254A;
N33Q+T231R+N233R+D254N;
N33Q+T231R+N233R+D254Q;
N33Q+T231R+N233R+D254I;
N33Q+T231R+N233R+D254L;
N33Q+T231R+N233R+D254K;
N33Q+T231R+N233R+D254M;
N33Q+S216P+L227G+T231R+N233R+Q249R;
D27V+N33Q+V60S+G91T+D96W+T231R+N233R+Q249R;
N33Q+D96N+L227G+T231R+N233R+Q249R;
D27R+N33Q+L227G+T231R+N233R;
D27R+N33Q+L227G+T231R+N233R+Q249R;
N33Q+E219D+L227G+T231R+N233R+Q249R;
D27Q+N33Q+L227G+T231R+N233R+Q249R;
N33Q+W117L+L227G+T231R+N233R+Q249R;
D5E+N33Q+W117L+L227G+T231R+N233R+Q249R;
D27Q+N33Q+E219D+L227G+T231R+N233R+Q249R;
N33Q+D96E+E219D+L227G+T231R+N233R+Q249R;
D27R+N33Q+E56K+G91N+N94R+D111A+S216P+L227G+T231R+N233R+P256T;
D27R+N33Q+E56Q+D57N+G91N+N94R+D111A+S216P+L227G+T231R+N233R+P256T;
D27R+N33Q+E56Q+D57N+G91N+N94R+D111S+S216P+L227G+T231R+N233R+D254S+P256T;
D27R+N33Q+E56S+G91N+N94R+D111A+S216P+L227G+T231R+N233R+P256T;
D27R+N33Q+G91N+N94R+D111A+S216P+L227G+T231R+N233R+D254S+P256T;
D27R+N33Q+G91N+N94R+D111A+S216P+L227G+T231R+N233R+D254S+P256T;
D27R+N33Q+G91N+N94R+D111S+A155V+S216P+L227G+T231R+N233R+D254S+P256T;
D27R+N33Q+G91N+N94R+D111S+S216P+L227G+T231R+N233R+D254S+P256T;
N33Q+D111A+T231R+N233R+D254S;
N33Q+D111A+W117L+T231R+N233R+D254S;
N33Q+T231R+N233R+P256A;
N33Q+T231R+N233R+P256N;
N33Q+T231R+N233R+P256G;
N33Q+T231R+N233R+P256H;
N33Q+T231R+N233R+P256L;
N33Q+T231R+N233R+P256M;
N33Q+T231R+N233R+P256S;
N33Q+T231R+N233R+P256W;
N33Q+T231R+N233R+P256Y;
N33Q+T231R+N233R+P256F;
N33Q+T231R+N233R+P256V;
N33Q+G91M+G163W+T231R+N233R;
N33Q+G91M+G163T+T231R+N233R;
N33Q+G91M+G163D+T231R+N233R;
N33Q+G91K+G163W+T231R+N233R;
N33Q+G91T+G163W+T231R+N233R;
N33Q+V176N+T231R+N233R;
N33Q+V176D+T231R+N233R;
N33Q+W117F+T231R+N233R;
N33Q+G91T+N94S+D111A+V176I+T231R+N233R+D254S;
N33Q+V176I+T231R+N233R;
N33Q+D111N+T231R+N233R;
N33Q+D111N+G225P+T231R+N233R;
N33Q+D111N+S216P+T231R+N233R;
D27R+N33Q+G91T+N94R+D111A+S216P+L227G+T231R+N233R;
N33Q+G91M+G163P+T231R+N233R;
N33Q+G91T+G163A+T231R+N233R;
N33Q+W117D+T231R+N233R;
N33Q+W117H+T231R+N233R;
N33Q+W117C+T231R+N233R;
N33Q+W117K+T231R+N233R;
N33Q+W117V+T231R+N233R;
N11S+N33Q+T231R+N233R;
N33Q+W117E+V176K+T231R+N233R;
N33Q+W117G+T231R+N233R;
N33Q+W117P+T231R+N233R;
N33Q+W117S+T231R+N233R;
N33Q+W117T+T231R+N233R;
N33Q+W117I+T231R+N233R;
D27R+N33Q+L227G+T231R+N233R+Q249R+D254S;
N33Q+S115L+T231R+N233R;
N33Q+G38A+G91T+G163K+T231R+N233R+D254S;
N33Q+V176M+T231R+N233R;
N33Q+V176H+T231R+N233R;
N33Q+V176A+T231R+N233R;
D27V+N33Q+L227F+T231R+N233R+Q249R;
N33Q+W117Y+T231R+N233R;
N33Q+W117Y+V176D+T231R+N233R;
D27V+N33Q+G91A+N94R+D111A+G163K+L227F+T231R+N233R+Q249R;
D27V+N33Q+G91A+N94R+D111A+G163K+L227F+T231R+N233R+Q249R+D254S;
D27R+N33Q+P136H+L227G+T231R+N233R+Q249R+D254S;
N11R+N33Q+T231R+N233R+T244S;
N33Q+G91T+D96N+D111A+V176I+T231R+N233R+D254S;
N33Q+G91T+N94S+D111A+V176I+T231R+N233R+D254S;
N33Q+G161A+T231R+N233R;
N33Q+G38I+G177A+T231R+N233R;
N33Q+N101Q+T231R+N233R;
N33Q+N94Q+T231R+N233R;
N33Q+G161A+T231R+N233R;
N11Q+N33Q+T231R+N233R;
N8Q+N33Q+T231R+N233R;
N33Q+T231R+N233R+N251Q;
N33Q+N200Q+T231R+N233R;
N33Q+G177A+T231R+N233R;
N33Q+N73Q+T231R+N233R;
N33Q+I86L+T231R+N233R;
N33Q+K98I+G163K+T231R+N233R;
D27R+N33Q+G91T+D96E+D111A+G163K+T231R+N233R+D254S+P256T;
D27R+N33Q+G91T+D96E+D111A+G163A+T231R+N233R+D254S+P256T;
D27R+N33Q+S216P+L227G+T231R+N233R+Q249R;
N33Q+K98I+G163K+N200Q+T231R+N233R+N251S;
N33Q+G38S+G163K+T231R+N233R;
D27R+N33Q+G38A+G91T+D96E+D111A+T231R+N233R+D254S+P256T;
N33Q G38Y T231R N233R;
D27R+N33Q+G91T+N94R+D111A+S216P+L227G+T231R+N233R+P256T;
D27R+N33Q+G91T+N94R+D111A+S216P+L227G+T231R+N233R+P256T;
N33Q+G38N+N73Q+T231R+N233R;
N33Q+G38D+R84E+T231R+N233R;
N33Q+G38Q+T231R+N233R;
N33Q+G38I+T231R+N233R;
N33Q+G38K+T231R+N233R;
N33Q+G38F+T231R+N233R;
N33Q+G38H+N200Q+T231R+N233R+N251S;
N33Q+G38L+T231R+N233R;
N33Q+G38M+T231R+N233R;
N33Q+G38F+T231R+N233R;
N33Q+G38P+T231R+N233R;
N33Q+G38T+T231R+N233R;
N11R+N33Q+G91T+W117I+G163K+T231R+N233R+D254S;
D27R+N33Q+G38A+G91T+D96E+D111A+G163K+T231R+N233R+D254S+P256T;
N11R+N33Q+G91T+W117I+G163K+T231R+N233R+D254S;
D27R+N33Q+G38A+G91T+D96E+D111A+G163A+T231R+N233R+D254S+P256T;
D27R+N33Q+V176Q+L227G+T231R+N233R+Q249R+D254S;
N33Q+W117I+V176Q+T231R+N233R+P256A;
N33Q+G38A+G163A+T231R+N233R+P256A;
N33Q+W117I+V176Q+T231R+N233R;
N33Q+G177A+T231R+N233R+G246A;
E1N+N33Q+T231R+N233R;
N33Q G38H T231R N233R;
N33Q+G91A+N94K+D111A+G163K+L227F+T231R+N233R+Q249R+D254S;
N11R+N33Q+G91T+G163K+V176Q+T231R+N233R+D254S;
N33Q+K98I+T231R+N233R;
D27R+N33Q+W117I+V176Q+L227G+T231R+N233R+Q249R+D254S;
N11R+N33Q+G38A+G91T+G163K+T231R+N233R+D254S;
N33Q+G163W+T231R+N233R;
N33Q+G38A+G163A+T231R+N233R;
D27R+N33Q+G91T+D96E+L97Q+D111A+T231R+N233R+D254S+P256T;
N33Q+T231R+N233R+D254Q;
N11R+N33Q+G91T+S115L+G163K+T231R+N233R+D254S;
N11R+N33Q+G91T+G163K+V176W+T231R+N233R+D254S;
N33Q+G163D+T231R+N233R;
N33Q+G163P+T231R+N233R;
E1D+N33Q+G91T+N94R+D111A+W117L+T231R+N233R+D254S;
N33Q+G91T+N94R+D111A+W117L+V176W+T231R+N233R;
Q4P+D27R+N33Q+G91N+N94R+D111A+L206F+S216P+L227G+T231R+N233R+P256T;
D27R+N33Q+T37K+N71I+G91N+N94R+K98I+D111A+S216P+L227G+T231R+N233R+P256T;
D27R+N33Q+E43K+K46M+I90V+G91N+N94R+D111A+T114I+S216P+L227G+T231R+N233R+P256T;
N33Q+W117S+T231R+N233R;
N33Q+G61R+V63R+G156R+V176W+T231R+N233R+P256I;
N33Q+D96N+G156R+V176W+T231R+N233R;
N33Q+G156R+V176W+T231R+N233R+Q249R;
N33Q+G91T+N94S+D111A+G163T+V176W+T231R+N233R;
N33Q+G91T+N94S+D111A+S115L+G163T+V176I+T231R+N233R;
N11R+D27R+N33Q+E56Q+D57N+G91N+N94R+D111S+G163T+S216P+L227G+T231R+N233R+D254S+P256T;
D27R+N33Q+E56Q+D57N+G91N+N94R+D111S+G163T+S216P+L227G+T231R+N233R+D254S+P256T;
N11R+D27R+N33Q+E56Q+D57N+G91N+N94R+D111S+S216P+L227G+T231R+N233R+D254S+P256T;
D27R+N33Q+E56Q+D57N+G91N+N94R+D111S+S216P+L227G+T231R+N233R+D242E+D254S+P256T;
D27R+N33Q+G38A+E56Q+D57N+G91N+N94R+D111S+S216P+L227G+T231R+N233R+D254S+P256T;
Q4R+D27Q+N33Q+G91T+N94S+E99D+D111A+E210D+S216P+L227G+T231R+N233R+P256L;
N33Q+G38A+G91T+G163A+T231R+N233R+D254S;
N33Q+G38A+G163A+T231R+N233R+D254I;
N11R+N33Q+I90L+G163L+T231R+N233R;
N11R+N33Q+I90L+G163L+T231R+N233R+D254S;
N11R+N33Q+E56Q+G91T+G163K+V176Q+T231R+N233R+D254S;
N11R+D27R+N33Q+G91T+D96E+D111A+G163K+T231R+N233R+D254S+P256T;
N11R+N33Q+G38A+G91T+G112A+G163A+T231R+N233R+D254S;
N11R+N33Q+G91T+G163K+E210D+T231R+N233R+D254S;
N11R+N33Q+G91T+G163K+T231R+N233R+D254I;
N11R+N33Q+G91T+G163K+V176T+T231R+N233R+D254S;
N11R+N33Q+G91T+G163P+T231R+N233R+D254S;
N11R+N33Q+G91M+G163T+T231R+N233R+D254S;
N11R+N33Q+G38A+G91T+G163K+V176D+T231R+N233R+D254S;
N33Q+E56Q+G156R+V176W+T231R+N233R;
E1D+N33Q+G38A+G91T+N94R+D111A+W117L+V176W+T231R+N233R;
N33Q+G163K+G177A+T231R+N233R+G246A;
N11R+N33Q+E56Q+G91T+G163K+T231R+N233R+D254S;
N11R+N33Q+I90L+G163K+T231R+N233R+D254S;
D27R+N33Q+E56Q+D57N+G91N+N94R+D111S+S216P+L227G+T231R+N233R+Q249R+D254S+P256T;
D27R+N33Q+E56Q+D57N+G91N+N94R+D111S+S216P+E219D+L227G+T231R+N233R+D254S+P256T;
N11R+N33Q+190L+G91T+N94S+D96E+G163K+T231R+N233R+D254S;
N11R+N33Q+G91T+G163K+V176I+T231R+N233R+D254S;
N11R+N33Q+G91T+G163K+V176Q+T231R+N233R+D254S;
N11R+N33Q+G91T+G163A+V176T+T231R+N233R+D254S;
N11R+N33Q+G91T+G163L+V176I+T231R+N233R+D254S;
N11R+N33Q+G91T+G163L+V176T+T231R+N233R+D254S;
N11R+N33Q+G91T+G163L+T231R+N233R+D254S;
N11R+N33Q+G91T+G163P+T231R+N233R+D254S;
N11R+N33Q+G91T+G163P+V176I+T231R+N233R+D254S;
N11R+N33Q+G91T+G163L+T231R+N233R+D254S+P256N;
D27R+N33Q+E56Q+D57N+G91N+N94R+D111S+G163T+S216P+L227G+T231R+N233R+Q249R+D254S+P256T;
Q4R+D27Q+N33Q+G91T+N94S+E99D+D111A+G163A+E210V+S216P+L227G+T231R+N233R+P256L;
Q4R+D27Q+N33Q+G91T+N94S+E99D+D111A+V176I+E210V+S216P+L227G+T231R+N233R+P256L;
N33Q+E210Y+T231R+N233R+D254Y+I255F;
N33Q+L93F+D102Y+T231R+N233R;
D27R+N33Q+L227G+T231R+N233R+Q249R+D254S;
N11S+N33Q+T231R+N233R;
N11R+N33Q+T231R+N233R;
N33Q+G38A+G91T+G163K+T231R+N233R+D254S;
N33Q+W117Y+V176T+T231R+N233R;
N8L+N11R+N33Q+G91T+G163K+T231R+N233R+D254S;
E1N+N33Q+G38A+G91T+G163P+V176F+T231R+N233R;
N11R+N33Q+G38A+G91T+G163P+V176G+T231R+N233R+D254S;
N11R+N33Q+G91T+G163K+T231R+N233R+D254A+P256F;
N11R+N33Q+G91T+G163K+T231R+N233R+P256F;
N11R+N33Q+G91T+G163K+T231R+N233R+D254S+P256F;
N11R+N33Q+G38A+G91T+G156R+G163K+V176T+T231R+N233R+D254S;
N33Q+G91K+D96S+G163T+T231R+N233R+Q249R;
N11R+N33Q+G91T+G163N+T231R+N233R+D254S;
N11R+N33Q+G91T+G163T+T231R+N233R+D254S;
N11R+N33Q+G91T+G163W+T231R+N233R+D254S;
N11R+N33Q+G91K+G163K+T231R+N233R+D254S;
N11R+G23E+N33Q+G91T+G163K+T231R+N233R+D254S;
N11R+N33Q+G91T+V141E+G163K+T231R+N233R+D254S;
N11R+N33Q+L52R+G91T+G163K+T231R+N233R+D254S;
N11R+N33Q+G91T+V141L+G163K+T231R+N233R+D254S;
N11R+N33Q+T37K+G91T+G163K+T231R+N233R+D254S;
N11R+N33Q+A68V+G91T+G163K+T231R+N233R+D254S;
N11R+N33Q+G91T+G163A+V176I+T231R+N233R+D254S;
N11R+N33Q+T37M+G91T+G163P+V176T+T231R+N233R+D254S;
N11R+N33Q+G91T+G163L+T231R+N233R+D254S;
N11R+N33Q+G91T+G163K+T231R+N233R+D254S+P256I;
N33Q+G38S+G156R+G163K+V176W+T231R+N233R;
N11R+D27R+N33Q+E56Q+D57N+G91N+N94R+D111S+G163K+S216P+L227G+T231R+N233R+D254S+P256T;
N11R+N33Q+G38A+G91T+G163P+V176G+T231R+N233R+D254S;
N11R+N33Q+G38A+G91T+G163Q+V176G+T231R+N233R+D254S;
N11R+N33Q+G38A+G91T+G163T+V176G+T231R+N233R+D254S;
N11R+N33Q+G38A+G91T+N94R+G163P+V176G+T231R+N233R+D254S;
E1*+N11R+N33Q+G38A+G91N+N94R+G163P+V176G+T231R+N233R+D254S;
E1N+N11R+N33Q+G38A+G91T+G163P+V176F+T231R+N233R;
E1N+F10L+N11R+N33Q+G38A+G91T+G163P+V176F+T231R+N233R;
E1N+N33Q+G38A+G91T+G163P+V176F+T231R+N233R+D254S;
E1N+N33Q+G38A+G91T+D111A+G163P+V176F+T231R+N233R;
E1N+N33Q+G38A+G91T+G163P+V176F+L227F+T231R+N233R;
E1N+N11R+N33Q+G38A+G91T+D111A+G163P+V176F+T231R+N233R;
E1N+N33Q+G38A+G91T+G163P+V176F+L227F+T231R+N233R+D254S;
E1N+N33Q+G38A+G91T+G163P+V176F+T231R+N233R+D254S+I255A+P256Q;
E1N+N11R+N33Q+G38A+G91T+D111A+G163P+V176F+T231R+N233R+D254S;
N33Q+G156R+V176W+T231R+N233R+P256I;
N33Q+G91T+N94S+D111A+G156R+G163T+V176W+T231R+N233R;
N33Q+G91T+N94S+D111A+G156R+G163T+V176I+T231R+N233R;
N11R+N33Q+G38A+G91T+D102G+S115L+G163K+T231R+N233R+D254S+P256T;
N11R+N33Q+G38A+G91T+S115L+G163K+T231R+N233R+D254S+P256T;
E1N+N11R+N33Q+G91T+G163A+T231R+N233R+G246A+D254S;
N11R+D27R+N33Q+D57G+G91T+D96E+D111A+G163K+T231R+N233R+D254S+P256T;
N33Q+D96N+G156R+V176W+T231R+N233R+Q249R;
N33Q+I86F+L93F+D102Y+E210Y+L227F+T231R+N233R+D254Y+I255F+L269F;
N33Q+I86F+L93F+D102Y+E210Y+L227F+T231R+N233R+D254Y+I255F;
N11C+N33Q+G91T+G163K+T231R+N233R+D254S;
N11L+N33Q+G91T+G163K+T231R+N233R+D254S;
N11H+N33Q+G91T+G163K+T231R+N233R+D254S;
N11D+N33Q+G91T+G163K+T231R+N233R+D254S;
N11R+N33Q+G91T+D96W+G163K+T231R+N233R+D254S;
D27R+N33Q+G91T+D96E+L97Q+D111A+G163K+T231R+N233R+D254S+P256T;
N11P+N33Q+G91T+G163K+T231R+N233R+D254S;
Q4R+D27N+N33Q+G38A+G91T+N94S+E99D+D111A+V176I+E210V+S216P+L227G+T231R+N233R+P256L;
N11R+N33Q+E56Q+G163K+T231R+N233R+D254S;
N11R+N33Q+G91T+G163A+T231R+N233R+D254S;
N11R+N33Q+G91T+G163P+T231R+N233R+D254S;
N11R+N33Q+G91T+G163K+L227G+P229R+T231R+N233R+D254S;
N33Q+E87K+T231R+N233R;
N33Q+N94K+T231R+N233R;
N33Q+D96Y+T231R+N233R;
N33Q+K98I+T231R+N233R;
A30V+N33Q+K98I+T231R+N233R;
N33Q+E87K+D96E+T231R+N233R;
N26I+N33Q+T231R+N233R;
A30T+N33Q+T231R+N233R;
N33Q+G91V+T231R+N233R;
N33Q+G91A+T231R+N233R;
N33Q+G91V+L97M+T231R+N233R;
N33Q+K98I+T231R+N233R;
N33Q+L69I+G91E+T231R+N233R;
P29T+N33Q+T231R+N233R;
N33Q+G91V+T231R+N233R;
N33Q+K98I+T231R+N233R;
N33Q+G91E+T231R+N233R;
N33Q+N94K+T231R+N233R;
D27R+N33Q+G91N+N94R+K98I+D111A+N162S+S216P+L227G+T231R+N233R+P256T;
D27R+N33Q+T37K+N71I+G91N+N94R+K98I+D111A+S216P+L227G+T231R+N233R+P256T;
D27R+N33Q+N39S+G91N+N94R+D111A+S216P+L227G+T231R+N233R+P256T;
D27R+N33Q+176T+G91N+N94R+R108M+D111A+S216P+L227G+T231R+N233R+P256T;
D27R+N33Q+L52I+V60E+G91N+N94R+D111A+T114I+V168M+E210D+S216P+L227G+T231R+N233R+P256T;
Q4P+D27R+N33Q+G91N+N94R+D111A+R205I+L206F+S216P+L227G+T231R+N233R+P256T;
Q4H+D27R+N33Q+G91N+N94R+D111A+V154L+S216P+L227G+T231R+N233R+P256T;
D27R+N33Q+G91N+N94R+D111A+V154I+S216P+L227G+T231R+N233R+P256T;
D27R+N33Q+N71S+G91N+N94R+D111A+H135D+S216P+L227G+T231R+N233R+P256T;
D27R+N33Q+G91N+N94R+K98I+D111A+S216P+L227G+T231R+N233R+P256T;
D27R+N33Q+G91N+N94R+L97M+D111A+S216P+T226N+L227G+T231R+N233R+P256T+L269H;
D27R+N33Q+G91N+N94R+D111A+T114I+R179T+S216P+L227G+T231R+N233R+P256T;
D27R+N33Q+G91N+N94R+D111A+S216P+L227G+T231R+N233R G23E+D27R+N33Q+L52R+G91N+N94R+D111A+T114I+V141E+S216P+L227G+T231R+N233R+P256T;
D27R+N33Q+E43K+K46M+190V+G91N+N94R+D111A+T114I+S216P+L227G+T231R+N233R+P256T;
D27R+A30V+N33Q+G91N+N94R+G109A+D111A+G190D+S216P+L227G+T231R+N233R+P256T;
D27R+N33Q+A49T+G91N+N94R+D111A+Y138F+G163R+S216P+L227G+T231R+N233R+P256T;
N26H+D27R+N33Q+G91N+N94R+D111A+V154F+G190C+S216P+L227G+T231R+N233R+P256T;
N33Q+G91T+D96E+K98T+T114I+G163S+E210V+T231R+N233R+D254K+P256A;
N33Q+G91T+D96E+K98T+T114I+T231R+N233R+G163S;
N33Q+G91T+D96E+K98T+T114I+G163K+E210D+T231R+N233R;
N33Q+G91T+T114I+G163K+E210D+T231R+N233R+D254G+P256A;
D27R+N33Q+G91T+T114I+G163W+E210D+T231R+N233R;
D27N+N33Q+G91T+T114I+G163S+E210D+T231R+N233R+P256T;
N33Q+G91T+T114I+G163K+E210D+T231R+N233R;
N33Q+G38W+G91T+T114I+G163K+E210V+T231R+N233R;
N33Q+G38W+G91T+T114I+G163K+E210D+T231R+N233R+P256T;
D27I+N33Q+G91T+D96E+K98T+T114I+G163K+E210D+T231R+N233R+P256T;
N33Q+G91T+T114I+E210V+T231R+N233R+D254K+P256A;
N33Q+G91A+N94K+D111A+G163K+L227F+T231R+N233R+Q249R;
G23E+D27R+N33Q+L52R+G91N+N94R+D111A+T114I+V141E+S216P+L227G+T231R+N233R+P256T;
D27R+N33Q+E43K+K46M+190V+G91N+N94R+D111A+T114I+S216P+L227G+T231R+N233R+P256T;
N33Q+G91T+K98I+T114I+G163K+T231R+N233R+D254S;
N33Q+G91T+K98I+G163K+T231R+N233R+D254S+P256L;
N33Q+G91T+T114I+G163K+T231R+N233R+D254S+P256L;
G23E+D27R+N33Q+L52R+G91N+N94R+D111A+T114I+V141E+S216P+L227G+T231R+N233R+P256T; and
D27R+N33Q+E43K+K46M+I90V+G91N+N94R+D111A+T114I+S216P+L227G+T231R+N233R+P256T.

In another embodiment, the lipase to be used as recombinantly produced purified microbial lipase is a variant of a parent lipase, which

(a) has at least 50% identity to amino acids 1 to 269 of SEQ ID NO: 2; and

(b) has lipase activity; and

(c) comprises at least one substitution selected from the following substitutions: N26I, D27Q, D27R, D27Y, P29T, A30T, A30V, T32I, N33Q, N33T, N33Y, P42L, E43D, E43K, E43M, E43V, A49T, E56A, E56C, E56K, E56R, E56S, D57A, D57G, D57N, V60L, L69I, E87K, G91A, G91E, G91N, G91R, G91S, G91T, G91V, G91W, L93F, N94K, N94R, N94S, D96E, D96G, D96L, D96N, D96S, D96V, D96W, D96Y, L97M, L97Q, K98I, E99D, E99K, E99P, E99S, E99T, D111A, D111S, T114I, L147S, G163K, E210D, S216P, L227G, T231R, N233R, D234K, E239V, Q249R, N251S, D254N, P256T, G263Q, L264A, I265T, G266D, T267A, and L269N, wherein each position corresponds to a position of amino acids 1 to 269 of SEQ ID NO: 2.

In yet another embodiment, the lipase to be used as recombinantly produced purified microbial lipase is a variant of a parent lipase, which
(a) has at least 50% identity to amino acids 1 to 269 of SEQ ID NO: 2; and
(b) has lipase activity; and
(c) as compared to the sequence of SEQ ID NO: 2 comprises a set of substitutions selected from the following:
G91A+D96W+E99K+G263Q+L264A+I265T+G266D+T267A+L269N;
N33Q+D96S+T231R+N233R+Q249R;
D27R+G91A+D111A+S216P+L227G+P256T;
D27R+G91N+N94R+D111A+S216P+L227G+P256T;
D27R+G91T+N94S+D111A+S216P+L227G+P256T;
D27R+G91S+D111A+S216P+L227G+P256T;
D27R+G91T+D96N+D111A+S216P+L227G+P256T;
N33Q+G163K+T231R+N233R;
T32I+G91V+T231R+N233R;
K98I+T231R+N233R;
G91A+T231R+N233R;
G91V+T231R+N233R;
N33Y+G91W+N94K+T231R+N233R;
P42L+D57N+G91E+T231R+N233R;
K98I+T231R+N233R;
V60L+G91V+T231R+N233R;
D57G+L93F+T231R+N233R;
A49T+E56R+E87K+E99S+T231R+N233R;
E99T+T114I+D254N+T231R+N233R;
D27Y+E87K+D96L+E99P+T231R+N233R;
E43K+E56S+E87K+T231R+N233R;
E56S+E87K+D96L+E99D+T231R+N233R;
E56A+D57A+T114I+T231R+N233R;
G91E+T231R+N233R;
E56K+D96G+D111A+T231R+N233R;
E87K+D111S+T231R+N233R;
E43V+G91R+T231R+N233R;
E56S+E87K+T231R+N233R;
E87K+G91E+T231R+N233R;
D27Y+E87K+T231R+N233R;
E43M+E87K+D96L+E99P+T231R+N233R;
E56K+E87K+D111A+T231R+N233R;
E87K+E99P+T231R+N233R;
E87K+D96L+E99P+T231R+N233R;
E56C+E87K+T231R+N233R;
E56R+E87K+D96L+T231R+N233R;
E43D+E56A+D57A+E87K+D111A+T231R+N233R;
E56K+E87K+D96L+E99P+T231R+N233R;
E87K+L147S+T231R+N233R;
D27Y+E87K+D96L+E99P+T231R+N233R;
E43D+E87K+D96L+E99P+E239V+T231R+N233R;
E43K+E56A+E87K+D234K+T231R+N233R;
D96V+D111A+T231R+N233R; and
N33T+E43V+E56K+D96G+T231R+N233R.

Herein, the amino acids were abbreviated using the One-Letter-Symbols (e. g. S, P, I, R, etc.) and/or the Three-Letter-Symbols (e. g. Ser, Pro, Ile, Arg, etc.) as listed e. g. in Voet & Voet, Biochemistry, 3rd Edition, John Wiley & Sons Inc.

The term “allelic variant” and the parameter “identity” describing the relatedness between two amino acid sequences are used herein according to the definitions as set out in International Patent Application PCT/DK2006/00352, published as WO 2006/136159, and incorporated herein by reference.

Isolation, purification, and concentration of a lipase to arrive at a recombinantly produced purified microbial lipase as described herein may be carried out by conventional means. In one embodiment, the recombinantly produced purified microbial lipase as described herein can be prepared by recovering in a first step a recombinantly produced microbial lipase from a fermentation broth by conventional procedures including, but not limited to, centrifugation, filtration, extraction, spray-drying, evaporation, or precipitation; and afterwards in a second step, purifying the recovered recombinantly produced microbial lipase by one or more purification method(s) known in the art. Suitable purification methods may be selected from, but is not limited to, chromatography methods (e.g., ion exchange chromatography, affinity chromatography, hydrophobic chromatography, chromatofocusing, and size exclusion chromatography), electrophoretic procedures (e.g., preparative isoelectric focusing), differential solubility (e.g., ammonium sulphate precipitation), SDS-PAGE, crystallization methods, extraction methods (see, e.g., Protein Purification, J. -C. Janson and Lars Ryden, editors, VCH Publishers, New York, 1989), and from combinations of any of the foregoing purification techniques or methods. In one embodiment, crystallization and/or chromatography methods may be used for commercial scale preparations. In another embodiment, crystallization is used for commercial scale preparations.

In one embodiment, the microbial lipase of SEQ ID NO: 2 may be prepared on the basis of U.S. Pat. No. 5,869,438 (the '438 Patent) (in which SEQ ID NO: 1 is a DNA sequence encoding the lipase of SEQ ID NO: 2 as defined herein), viz. by recombinant expression of a DNA sequence of SEQ ID NO: 1 of the '438 patent in a suitable host cell.

In another embodiment, the lipase of SEQ ID NO: 1 may be prepared on the basis of the '438 Patent (in which SEQ ID NO: 1 is a DNA sequence encoding a similar lipase differing only in amino acid position numbers 231 and 233), viz. by recombinant expression in a suitable host cell of a DNA sequence which is a modification of SEQ ID NO: 1 of the the '438 Patent which reflects the two amino acid differences.

In a further embodiment, the microbial lipase variants of SEQ ID NO: 1 and/or SEQ ID NO: 2 as preferably used herein may, e.g., be prepared based on the '438 Patent (in which SEQ ID NO: 1 is a DNA sequence encoding the lipase of SEQ ID NO: 2 as defined herein), viz. by recombinant expression of a DNA sequence in a suitable host cell, which DNA sequence is a modification of SEQ ID NO: 1 of the '438 Patent, the modification reflecting the amino acid differences between the desired lipase variant and the lipase of SEQ ID NO: 2 herein. Such modifications can be made by site-directed mutagenesis, as is known in the art.

In one embodiment, the microbial lipase variants of SEQ ID NO: 1 and/or SEQ ID NO: 2 are prepared by transforming the DNA encoding the lipase variants into Aspergillus oryzae strain ToC1512 (described in WO2005070962 A1), using the method described in Example 22 of the '438 Patent except that PyrG selection is used (described in WO2004069872 A1) instead of AMDS selection. Spores of the Aspergillus oryzae host are taken from an agar slant and used for inoculation of 10 ml YPM (10 g yeast extract, Difco+20 g Peptone, Difco, water to 1 L, is autoclaved; add sterile filtered maltose to 2% (w/w)). Inoculated tubes are incubated at 30° C. for three days in a New Brunswick Scientific Innova 2300 shaker at 180 rpm. Supernatants are harvested by filtering cultures with Mira-Cloth (Calbiochem) followed by sterile filtration with 0.45 um (micro meter) filters. The lipase variants are further purified as generally described in Example 23 of the '438 Patent.

In other embodiments, concentrated solid or liquid preparations of each of the recombinantly produced purified microbial lipases are prepared.

In a further embodiment, the recombinantly produced purified microbial lipase(s) are used in the form of solid concentrates. The recombinantly produced purified microbial lipase(s) can be brought into the solid state by various methods as is known in the art. In one embodiment, the solid state can be either crystalline, where the lipase molecules are arranged in a highly ordered form, or a precipitate, where the lipase molecules are arranged in a less ordered, or disordered, form. Various precipitation methods are known in the art, including precipitation with salts, such as ammonium sulphate, and/or sodium sulphate; with organic solvents, such as ethanol, and/or isopropanol; or with polymers, such as PEG (Poly Ethylene Glycol). In an alternative embodiment, the lipase(s) can be precipitated from a solution by removing the solvent (typically water) by various methods known in the art, e.g., lyophilization, evaporation (for example at reduced pressure), freeze-drying and/or spray drying.

In another embodiment, crystallization can be used as a method to purify a lipase to arrive at a recombinantly produced purified microbial lipase. Crystallization may, for example, be carried out at a pH close to the isoelectric point (“pI”) of the lipase(s) and at low conductivity, for example 10 mS/cm or less, as described in EP 691982. In one embodiment, the lipase is a crystalline lipase, which can be prepared as described in Example 1 of EP 600868 B1. In another embodiment, the lipase crystals may furthermore be cross-linked as described in WO 2006/044529.

In one embodiment, the solid concentrate of the lipase(s) has a protein purity of active enzyme protein of at least 50% (w/w) by reference to the total protein content of the solid concentrate. In still further embodiments, the protein purity of the active enzyme protein relative to the total protein content of the solid concentrate is at least 55, 60, 65, 70, 75, 80, 85, 90, or at least 95% (w/w). The protein purity can be measured as is known in the art, for example by densitometer scanning of coomassie-stained SDS-PAGE gels, e.g., using a GS-800 calibrated densitometer from BIO-RAD; by using a commercial kit, such as Protein Assay ESL, order no. 1767003, which is commercially available from Roche; or on the basis of the method described in Example 8 of WO 01/58276. In one embodiment, the lipase enzyme protein constitutes at least 50% of the protein spectrum of the solid lipase concentrate for use according to the present disclosure, as measured by densitometer scanning of a coomassie-stained SDS-PAGE gel. In further embodiments, the lipase enzyme protein constitutes at least 55, 60, 65, 70, 75, 80, 85, 90, 92, 94, 95, 96, or at least 97% of the protein spectrum of the solid lipase concentrate for use according to the present disclosure, as measured by densitometer scanning of a coomassie-stained SDS-PAGE gel. Such enzymes may be designated “isolated”, “purified”, or “purified and isolated” enzymes or polypeptides. For the lipase expressed in Aspergillus and comprising a mixture of the various N-terminal forms of SEQ ID NO: 1 as explained in Example 5 of WO 2006/136159, the relevant band on an SDS-PAGE gel corresponds with a molecular weight of 34-40 kDa. For the non-glycosylated variant of SEQ ID NO: 1, N33Q, the relevant band is located at around 30 kDa.

In one embodiment, a recombinantly produced purified microbial lipase is produced from a recombinantly produced microbial lipase, in particular, a lipase from Humicula lanuginosa. For this purpose, a recombinantly produced microbial lipase, in particular a lipase from Humicula lanuginosa, is recovered from a fermentation broth by a conventional procedure as described above and is obtained as a liquid lipase concentrate. A solid lipase concentrate is produced from said liquid lipase concentrate by a conventional precipitation or drying process, such as spray-drying. In one embodiment where a lipase from Humicula lanuginosa is used, the solid lipase concentrate obtained by the described method has typically a protein content of about 50% (w/w) and a protein purity of about 95 area-%. Said solid lipase concentrate may be purified further by conventional methods as desired or needed.

In another embodiment where a lipase from Humicula lanuginosa is used, further purification of the solid lipase concentrate by crystallization as described above is preferred. In a further embodiment, the solid lipase concentrate from Humicula lanuginosa can be crystallized for purification in a suitable crystallization buffer at a pH close to its pI and at low conductivity. The crystallized lipase may then be separated from the crystallization buffer by conventional separation processes, such as centrifugation, and may be re-dissolved at a higher pH. If desired, further purification process cycles may be carried out to arrive at a specified or desired protein purity and/or protein content. The purified liquid lipase concentrate such obtained can then be transformed into a purified solid lipase concentrate by conventional precipitation or drying processes, such as spray-drying. The solid lipase concentrates and the purified solid lipase concentrates may themselves be suitable as purified lipases according to this disclosure, depending on their protein contents and/or protein purities.

A recombinantly produced purified microbial lipase which is used according to the present disclosure can expediently have a residual moisture content of 1% to 7% determined according to conventional methods, such as the method of Karl Fischer as described in U.S. Pat. No. 6,355,461.

It has now been found that recombinantly produced purified microbial lipases with a protein content of below 60% (w/w), e.g., about 50% (w/w), can be processed into suitable pharmaceutical administration forms by conventional extrusion techniques without a significant loss of protein purity in the purified lipase used. Where, however, recombinantly produced microbial purified lipases with a higher protein content of at least 60% (w/w) are used, e.g., a protein content of 80% (w/w), these are not typically able to be processed into suitable pharmaceutical administration forms by conventional extrusion techniques without a significant loss of protein purity after processing.

In the present disclosure, a lipase reference standard was used which shows a very high purity. In one embodiment, a lipase reference standard was used which shows the highest available purity and nearly the maximum specific activity (or approximate specific activity, see above). In another embodiment, a lipase reference standard was used which shows a maximum specific activity for the respective lipase. For each recombinantly produced purified microbial lipase, a lipase reference standard was prepared wherein the amino acid sequence of the lipase reference standard is the same as for the recombinantly produced purified microbial lipase, i.e., both are the same lipases but have been purified by different methods:

a) Manufacturing of the Recombinantly Produced Purified Microbial Lipase
The unpurified lipase (e.g., as obtained by fermentation) is purified by crystallization technology at a defined pH value as described herein.

b) Manufacturing of the Lipase Reference Standard
The unpurified lipase (e.g., as obtained by fermentation) is purified using the most efficient purification method currently known in the art. In a one embodiment, the lipase is purified using chromatography methods; in a another embodiment, using three combined chromatography methods comprising hydrophobic interaction chromatography (HIC), ion exchange chromatography and size exclusion chromatography (SEC); in a further embodiment, using purification in a first step by HIC, in a second step by ion exchange chromatography and in a third step by SEC; to achieve a lipase of very high purity, and in one embodiment to achieve a lipase of the highest available purity.

In one embodiment, the lipase reference standard is prepared by the following process: The starting material is suspended in a suitable liquid, such as water, and in one embodiment, water adjusted to a defined pH, such as to pH 6. A defined volume of buffer medium, such as a defined volume of succinic acid/NaOH solution and a defined volume of a dissolved osmotic active agent, such as a defined volume of a NaCl solution, are added and the pH is adjusted to a suitable pH, such as pH 6. Afterwards, the mixture is filtered through a suitable filtration unit, such as a 0.22 μm filtration unit. A defined volume of the filtrate is applied to a suitable separation column, such as a suitable hydrophobic interaction chromatography separation column, and in one embodiment a acetylated decylamin-agarose (decyl-agarose) column, which is equilibrated in a suitable equilibration buffer with a suitable pH, such as in a solution of succinic acid NaOH/solution, NaCl with a suitable pH, such as with a pH of 6. The column is washed with the equilibration buffer. Subsequently, the column is stepwise eluted with a suitable elution liquid with a suitable pH, such as a H₃BO₃/NaOH solution containing isopropanol with a suitable pH, such as a pH of 9. This step is repeated a defined number of times, such as 19 times (20 times in total). All the eluates are combined and diluted to a defined volume with a suitable liquid, such as water. The diluted lipase is applied to a suitable separation column, such as a suitable ion exchange chromatography column, and in one embodiment a Q-sepharose FF column, equilibrated in a suitable equilibration buffer, such as a H₃BO₃/NaOH solution with a suitable pH, such as a pH of 9. The column is washed with the equilibration buffer. Subsequently the column is eluted with a suitable elution liquid, such as a linear gradient liquid, and in one embodiment a linear NaCl gradient (0→0.5M) over a suitable number of column volumes, such as 3 column volumes. The eluted lipase peak is transferred to a suitable solution, such as a HEPES/NaOH, NaCl solution, CaCl₂ solution, with a suitable pH, such as a pH of 7, by buffer exchange on a suitable separation column, such as a size exclusion chromatography separation column, and in one embodiment a sephadex G25 column. The buffer exchanged lipase is filtered through a suitable filtration unit, such as a 0.22 μm filtration unit. The lipase solution obtained by this process is used as lipase reference standard. The lipase reference standard is characterized protein purity, protein content and specific activity. A lipase reference standard obtained by this purification process shows a high or a very high purity. In one embodiment, the lipase reference standard shows a purity of higher than 99.9% (i.e. less than 0.1% impurities).

Another embodiment relates to a pharmaceutical composition comprising the granules containing recombinantly produced purified microbial lipase and optionally further conventional pharmaceutical auxiliaries and/or excipients. For said pharmaceutical compositions, the granules comprising purified lipase can be used alone or in combination with appropriate conventional pharmaceutical auxiliaries and/or excipients, such as conventional carriers, such as lactose, mannitol, corn starch, or potato starch; with excipients, such as crystalline cellulose or microcrystalline cellulose, cellulose derivatives, acacia, corn starch, or gelatins; disintegrants, such as corn starch, potato starch, or sodium carboxymethylcellulose; lubricants, such as carnauba wax, white wax, shellac, waterless colloid silica, polyethylene glycol (PEGs, also known under the term macrogol) from 1500 to 20000, in particular PEG 4000, PEG 6000, PEG 8000, povidone, talc, monolein, or magnesium stearate; and if desired, with further auxiliaries and/or excipients like diluents, adjuvants, buffering agents, moistening agents, preservatives such as methylparahydroxybenzoate (E218), colouring agents such as titanium dioxide (E171), and/or flavouring agents like saccharin, orange oil, lemon oil, and/or vanillin. In further embodiments, conventional pharmaceutical auxiliaries and/or excipients according to the present disclosure may be selected from material such as (i) one or more carriers and/or excipients; or (ii) one or more carriers, excipients, diluents, and/or adjuvants.

Generally, depending on the medical indication in question, the pharmaceutical composition may be designed for all manners of administration known in the art, including enteral administration (through the alimentary canal) and oral administration. In one embodiment, the method of administration is oral. Thus, the pharmaceutical composition is usually in solid form, such as capsules, granules, micropellets, microtablets, pellets, pills, powders, microspheres and/or tablets. Capsules, granules, microtablets, pills, powders and/or tablets are preferred. For the purposes of this disclosure, the prefix “micro” is used to denominate an oral dosage form if the diameter of the oral dosage form or all of its dimensions (length, height, breadth) is equal to or below 5 mm. The medical practitioner will know to select the most suitable route of administration and avoid potentially dangerous or otherwise disadvantageous administration routes.

According to a further embodiment, the inventive pharmaceutical composition may optionally be further incorporated in one or more packages selected from the group consisting of sachets, blisters or bottles.

In one embodiment, the oral dosage form is a capsule which contains the pharmaceutical composition comprising granules of the present disclosure. These granules consist of: (1) 10-90% by weight of recombinantly produced purified microbial lipase; (2) 1-50% by weight of sucrose, (3) 0-25% by weight of hypromellose, and (4) 10-90% by weight of non-pareil beads consisting of microcrystalline cellulose. In further embodiment, the micropellets or microspheres consist of: (1) 20-50% by weight of recombinantly produced purified microbial lipase; (2) 5-25% by weight of sucrose, (3) 0-5% by weight of hypromellose, and (4) 30-60% by weight of non-pareil beads consisting of microcrystalline cellulose.

The amount of recombinantly produced purified microbial lipase in a pharmaceutical composition may vary within the group of lipases suitable to be used in the context of the present disclosure. In general, the amount of recombinantly produced purified microbial lipase in the resulting inventive pharmaceutical composition or medicament must be therapeutically effective to the prevention or treatment of diseases and disorders, such as diseases and disorders selected from the group consisting of: digestive disorders, pancreatic exocrine insufficiency, pancreatitis, cystic fibrosis, diabetes type I and/or diabetes type II. Examples of anticipated daily clinical dosages are as follows (all in mg purified lipase protein per kg of bodyweight): 0.01-1000, 0.05-500, 0.1-250, 0.5-100 or 1.0-50 mg/kg bodyweight.

Yet another embodiment relates to the novel pharmaceutical composition comprising granules containing recombinantly produced purified microbial lipase, for use as a medicament, in particular a medicament for the prevention or treatment of diseases and disorders, such as digestive disorders, pancreatic exocrine insufficiency, pancreatitis, cystic fibrosis, diabetes type I and/or diabetes type II.

A further embodiment relates to the novel pharmaceutical composition comprising granules containing recombinantly produced purified microbial lipase for the prevention or treatment of diseases and disorders, such as digestive disorders, pancreatic exocrine insufficiency, pancreatitis, cystic fibrosis, diabetes type I and/or diabetes type II.

A yet further embodiment relates to a method of preventing or treating diseases and disorders, such as digestive disorders, pancreatic exocrine insufficiency, pancreatitis, cystic fibrosis, diabetes type I and/or diabetes type II by administering to a mammal, in particular a human, in need thereof a therapeutically effective amount of (i) either a recombinantly produced purified microbial lipase which has a protein purity of at least 90 area-% (w/w) and a protein content of at least 60% (w/w), or (ii) a pharmaceutical composition as described herein.

The use of microbial derived enzymes also allows individual dosing of the respective enzymes. By using a suitable device for each enzyme, the dosage can be adapted to the individual needs of a particular patient, patient population or patient sub-population. Where, e.g., the physiological condition of a given patient requires the administration of high amounts of lipase activity, more lipase containing granules can be dispensed whereas the number of protease and/or amylase containing granules (and thus the protease and/or amylase activities) which is/are administered remain(s) the same. In one embodiment, the suitable device is a device for dosing. In a further embodiment, the suitable device is a common dispenser for pharmaceutical use.

According to the present disclosure, the embodiments of the pharmaceutical composition comprising granules, in particular, of the core particles and the coating and layers, are also applicable for pharmaceutical compositions.

A further embodiment relates to a process for the manufacture of novel pharmaceutical composition comprising granules containing purified lipase, comprising or consisting of the steps of:
a) providing pharmaceutically acceptable core particles,
b) providing a coating solution comprising at least one recombinantly produced purified microbial lipase which has a purity of at least 90 area-% and a protein content of at least 60% (w/w),
c) coating one or more times the core particles of step a) with the coating solution of step b) to obtain granules containing at least one recombinantly produced purified microbial lipase, and
d) optionally incorporating the granules of step c) into a suitable pharmaceutical composition.
   In process step a), pharmaceutically acceptable core particles as described above are provided.

The coating solution of step b) is obtained by dispersing or solving the solid form of the recombinantly produced purified microbial lipase in a solvent suitable for the purpose, such as water, and in one embodiment purified (pharmaceutical grade) water. In one embodiment, only one recombinantly produced purified microbial lipase is used in process step b). In another embodiment, one or more enzyme stabilizing agents and/or one or more binding agents, both as described above, may be added to the suspension or solution. If desired, additional pharmaceutical auxiliaries and/or excipients may also be added. Where necessary, the coating solution comprising recombinantly produced purified microbial lipase is stored at such cool temperature that the enzyme activity is not negatively affected and microbial growth is suppressed. In one embodiment, the coating solution is stored at approximately 0° C. to 10° C., 2° C. to 8° C., or approximately 5° C.

In one embodiment of the manufacturing process, the coating step c) is carried out in a coating chamber, such as a fluid bed apparatus. Where a fluid bed coater is used, this may, e.g., be a Wurster apparatus. In one embodiment, a fluid bed coater is equipped with a two-fluid-nozzle. The required or desired amount of core particles are then weighed and placed into the reaction chamber in a manner known per se, and the core particles are preheated to temperature suitable for coating.

The core particles are then coated in step c) by spraying the recombinantly produced purified microbial lipase comprising solution from step b) onto the core particles in a manner known per se, whereby the temperature of the recombinantly produced purified microbial lipase comprising solution is preferably kept at such a temperature or temperature range that the enzyme activity is not negatively affected, i.e., the temperature is usually kept below 100° C. In another embodiment, the temperature is kept below 90° C.

The product temperature of the coated granules is preferably controlled not to exceed a temperature where the enzyme activity of the recombinantly produced purified microbial lipase is negatively affected. Accordingly, the temperature of the coated granules is controlled not to exceed a temperature range of approximately 30° C. to 90° C., approximately 45° C. to 70° C., or approximately 49° C. The product temperature may be controlled in a manner known per se, e.g., by the drying air temperature.

Once all the desired coating solution comprising recombinantly produced purified microbial lipase of step b) has been sprayed onto the core particles, the heating element of the reaction chamber is preferably turned off and the process is stopped. If necessary, the resulting granules can subsequently be dried in conventional manner. Where desired, the coating process may be repeated once or more times to apply one or more additional coating layers to the core particles. The additional coating layers may comprise the same or different recombinantly produced purified microbial lipase(s). In one embodiment, the additional coating layers comprise the same recombinantly produced purified microbial lipase. To arrive at a coating layer of desired thickness, the coating process may be performed continuously or discontinuously.

A further embodiment relates to pharmaceutical compositions comprising or consisting of granules containing recombinantly produced purified microbial lipase, said pharmaceutical compositions being obtainable by the process for the manufacture of novel pharmaceutical compositions as described herein.

Various references are cited herein, the disclosures of which are incorporated herewith by reference in their entireties.

### Examples

1. Recovery and Purification of a Recombinantly Produced Microbial Lipase

a) Recovery of a Lipase from Humicula lanuginose

The lipase of SEQ ID NO: 1 is expressed in Aspergillus oryzae and purified from the fermentation broth as described in Example 22 and 23 of U.S. Pat. No. 5,869,438. The lipase is identified as the main protein band at approximately 30 kDa. By densitometer scanning of coomassie-stained SDS-PAGE gels, this band is found to constitute 92-97% of the protein spectrum. The densitometer is a GS-800 calibrated densitometer from BIO-RAD. The characterization of this protein band is performed as described in Example 11.

The liquid lipase concentrate obtained is spray dried to obtain a solid lipase concentrate.

The specific activity of the recovered solid lipase concentrate can be determined as described in Example 8. It is at least 1,000,000 U/g

The protein content of the recovered solid lipase concentrate is determined as described in Example 6. The protein content is at least 50% (w/w).

The protein purity of the recovered solid lipase concentrate is determined as described in Example 10. The protein purity is about 94 area-%.

b) Purification of a Lipase from Humicula lanuginose

The lipase obtained in step a) above is crystallized at a pH close to its pI and at low conductivity prior to drying. The crystals are repeatedly isolated by centrifugation, washed with a buffer solution and again isolated by centrifugation (2-3 times in total). The pH is increased by adding NaOH to dissolve the lipase crystals and the resulting purified liquid lipase concentrate is filtered if desired.

The purified liquid lipase concentrate obtained is spray dried to obtain a purified solid lipase concentrate.

The protein content of the recovered purified solid lipase concentrate is determined as described in Example 6. The protein content is about 80% (w/w).

The protein purity of the recovered purified solid lipase concentrate is determined as described in Example 10. The protein purity is about 99 area-%.

2. Preparation of Granules According to the Present Disclosure from a Recombinantly Produced Purified Microbial Lipase

900 g of sucrose and 150 g of hypromellose are weighed and stirred into 17 kg purified water. A 3-kg-fraction of the recombinantly produced purified microbial lipase (=purified solid lipase concentrate) as obtained from Example 1 is sieved using a 0.71 mm sieve and stirred into the prepared sucrose/hypromellose solution. A fluid bed coater equipped with a two-fluid nozzle and a wurster apparatus is preheated to approximately 50° C. An amount of 3 kg of microcrystalline cellulose pellets of an average diameter of 500 μm are weighed and placed into the fluid bed coater and are preheated to a product temperature of approximately 50° C. The pellets are coated by spraying the solution of purified lipase on the pellets in a manner known per se. The solution of recombinantly produced purified microbial lipase is kept at 5±3° C. during spraying. The product temperature is controlled not to exceed 55° C., preferentially being approximately 49° C. by controlling the drying air temperature. Once all solution of recombinantly produced purified microbial lipase is sprayed onto the pellets, the heater of the fluid bed dryer is turned off and the process is stopped after another five minutes. The product is packed and tested.

3. Encapsulation of Pellets which are Coated with Recombinantly Produced Purified Microbial Lipase (50 mg)

The required amount of pellets which are coated with recombinantly produced purified microbial lipase (resulting in granules) to be filled into capsules is calculated according to the following formula:

Filling weight=50 mg×1000/lipase protein content of granules (mg/g)

The calculated amount of pellets is encapsulated into hard gelatin capsules, size 2. The product is packed and tested. Capsules can also be filled with granules which are coated according to Example 12 or 13.

4. Production of Pellets Containing Recombinantly Produced Purified Microbial Lipase by an Extrusion Process not According to the Present Disclosure

750 g of recombinantly produced purified microbial lipase from Example 1 (=purified solid lipase concentrate) and 750 g of microcrystalline cellulose are dry-premixed in a mixer. After addition of 1171 g isopropanol, 70% of the mass is mixed and extruded with a conventional extruder through a die with holes of 0.8 mm diameter to form cylindrical pellets. The bead temperature does not exceed 50° C. while pressing. The extrudate produced is rounded to spherical pellets with a conventional spheronizer by adding the necessary amount of isopropyl alcohol (70%). The pellets are dried at a supply temperature of approximately 40° C. in a vacuum dryer (product temperature not to exceed 45° C.). Separation of the dried pellets is performed using a mechanical sieving machine with 0.7 and 1.4 mm screens. The sieve fraction of ≧0.7 mm and ≦1.4 mm are collected for further processing. Over- and undersized pellets are rejected and kept for further use.

5. Comparison of Granules

Comparative experiments were performed to determine the lipase activity and protein purity obtained when preparing i) pellets containing recombinantly produced purified microbial lipase by an extrusion process and ii) granules containing recombinantly produced purified microbial lipase by coating core particles.

Pellets containing recombinantly produced purified microbial lipase were manufactured using an extrusion process (as described in Example 4). Granules containing recombinantly produced purified microbial lipase were manufactured by coating core particles (as described in Example 2). The activity of the recombinantly produced purified microbial lipase was determined in each of pellets and granules as described in Example 7. The protein purity of the recombinantly produced purified microbial lipase was determined as described in Example 10.

TABLE 1
Comparison of lipase activity and protein purity in pellets and in granules

| | | | Granules |
|---|---|---|---|
| | Purified lipase | Pellets | prepared by |
| | (starting | prepared by | coating core |
| | material) | extrusion | particles |
| Lipase Activity | — | 94.1 | 96.4 |
| (% recovery, | | | |
| corrected for | | | |
| process yields) | | | |
| Protein purity (%) | 99.9 | 97.1 | 99.6 |
| (HPLC) | | | |

As can be seen from Table 1, the recombinantly produced purified microbial lipase granules show a higher activity and a higher purity in comparison to the recombinantly produced purified microbial lipase pellets manufactured by an extrusion process.

6. Determination of the Protein Content of the Recombinantly Produced Purified Microbial Lipase Using RP-HPLC

The protein content of the recombinantly produced purified microbial lipase from Example 1 is determined by gradient RP-HPLC with acetonitirile/water/TFA at a detection wavelength of 214 nm. The separation was performed on a YMC Protein RP, S-5 μm column, 125×3 mm I.D. (YMC Europe GmbH, Schermbeck, Germany) by running a gradient from 0 to 90% acetonitrile/TFA 0.05% within 50 minutes at a flow rate of 1.0 ml/min. The sample to be examined was to be dissolved in an aqueous solution of sodium chloride 2% w/w. The column was operated at 40° C.

The assaying of the lipase protein content was performed by the external standard method. A well characterized lipase reference standard was used as reference where the absolute protein content had been determined independently by amino acid analysis (assaying the content of amino acids after hydrolysis by HPLC after derivatization). Quantification of all peaks is performed according to the area-% method and the area-% of the lipase peaks are expressed as percentage of the total area.

7. Determination of the Lipase Activity

The lipolytic activity is determined by an enzymatic assay based on hydrolysis of olive oil by lipase and titration of the fatty acids released as follows:

As a substrate for the enzymatic assay, olive oil (175 g) is mixed with 630 mL of a solution of acacia gum (474.6 g gum arabic, 64 g calcium chloride in 4000 mL water) for 15 min in a blender to obtain an emulsion. After cooling to room temperature, pH is adjusted to 6.8 to 7.0 using 4M NaOH. For the lipase activity determination, 19 mL of the emulsion and 10 mL bile salt solution (492 mg bile salts are dissolved in water and filled up to 500 mL) are mixed in the reaction vessel and heated to 36.5° C. to 37.5° C. Reaction is started by addition of 1.0 mL of enzyme solution. The released acid is titrated automatically at pH 7.0 by addition of 0.1M sodium hydroxide for a total of 5 min. The activity is calculated from the slope of the titration curve between the 1^{st} and the 5^{th} minute. For calibration, a standard is measured at three different levels of activity. This reference standard has a defined absolute activity where 1 unit is defined as the enzymatic activity which hydrolyses 1 μequivalent of acid within one minute at a pH of 7.0 at 37° C.

8. Determination of the Specific Activity of the Recombinantly Produced Purified Microbial Lipase

The specific activity is calculated from the ratio of the lipolytic activity determined by titration (see Example 7) over the protein content as determined by HPLC (see Example 6) in lipase units/g (U/g).

9. Determination of the Enzyme Activity Based on the Total Weight of the Composition

The enzyme activity is calculated from the ratio of the lipolytic activity determined by titration (see Example 7) either over the total weight of the granules contained in the inventive pharmaceutical composition (manufactured as described in Example 2) or over the total weight of extrusion pellets (manufactured as described in Example 4) as determined by conventional methods.

10. Determination of the Protein Purity

The protein purity of a lipase preparation or a recombinantly produced purified microbial lipase is determined by a chromatographic method. To this purpose, the percentage of peptidic impurities is assayed by using the same HPLC method as for assaying the protein content (see Example 6). The peptidic impurities are separated from the main compound lipase and are calculated as peak area-%.

11. Characterization of the Recombinantly Produced Purified Microbial Lipase

The following slightly different N-terminal forms of SEQ ID NO: 1 are identified by N-terminal sequencing of this main protein band (see Example 1), below listed according to abundance. The amount of the various forms is determined by N-terminal sequencing by comparing the initial yields of the different forms in the first cycle of Edman degradation. The yields of the five N-terminal forms in the samples are also indicated:

| | | |
|---|---|---|
| #1 SPIRREVSQDLF . . . | (amino acids −5-269 of SEQ ID NO: 1) | 45-65% |
| #2 EVSQDLF . . . | (amino acids 1-269 of SEQ ID NO: 1) | 35-47% |
| #3 VSQDLF . . . | (amino acids 2-269 of SEQ ID NO: 1) | <1% to 16% |
| #4 PIRREVSQDLF . . . | (amino acids −4-269 of SEQ ID NO: 1) | <1% |
| #5 IRREVSQDLF . . .. | (amino acids −3-269 of SEQ ID NO: 1) | <1% |

The two major forms #1 and #2 are found in all batches, form #3 in some batches but not all, and forms #4 and #5 in very low amounts in some batches (close to or below the detection limit).

It is believed that these variants have been formed as a result of cleavage by endogenous Aspergillus host proteases. For example, #2 might have been formed due to cleavage of #1 by KexB protease, #3 by cleavage with KexB and afterwards by aminopeptidase, and #4 and #5 by cleavage with aminopeptidase.

The quantification based on N-terminal sequencing is confirmed by Electro Spray Ionization Mass Spectrometry (“ESI-MS”), which showed matching mass intensities. The difference between #1, #2, and #3 result in different theoretical pI values of 5.45, 5.11 and 5.3, respectively. Accordingly, these three forms are separated by IEF (IsoElectric Focusing), viz. on a pH 3-7 IEF gel. The bands are confirmed by N-terminal sequencing of blotted IEF gels. IEF is accordingly an easy and fast method for detection and quantification of forms #1, #2, and #3 of SEQ ID NO: 1.

Forms #1 and #2 of SEQ ID NO: 1 are found to have the same specific activity in LU/g enzyme protein. Specific lipase activity is determined as described in Example 7 and Example 8.

Amino Acid Analysis (“AAA”)/(mg/ml): The peptide bonds of the lipase sample are subjected to acid hydrolysis, followed by separation and quantification of the released amino acids on a Biochrom 20 Plus Amino Acid Analyser, commercially available from Bie & Berntsen A/S, Sandbaekvej 5-7, DK-2610 Roedovre, Denmark, according to the manufacturer's instructions. The amount of each individual amino acid is determined by reaction with ninhydrin.

ESI-MS data of the various lipase batches also clearly show a complex glycosylation pattern corresponding to high mannose glycosylation with a number of mass peaks separated by a molecular weight corresponding to one hexose.

SEQ ID NO:1 includes one putative N-glycosylation site (NIT), N being residue number 33 of SEQ ID NO: 1. In fungal expression hosts, N-acetylglucosamine residues will be linked to N-residues in a NIT-sequence as a result of post-translational modification, and a number of mannose monomers (from 5 to 21) will in turn be attached to the N-acetylglucosamine residues. This leads to a great variation in molecular weight of individual glycosylated molecules. By ESI-MS, the molecular weight ranges from approximately 30-34 kDa. The molecular weight of a typical iso-form (2 N-acetyl hexoses+8 hexoses) of the full length glycosylated protein has been determined as 31,721 Da by ESI-MS. The theoretical molecular weights of #1 and #2 without glycosylation are 30.2 kDa, and 29.6 kDa, respectively. This means that when expressed in a non-glycosylating host, the main band on an SDS-PAGE gel will be narrower and corresponding to a molecular weight of around 30 kDa. The molecular weight of the full length de-glycosylated protein has been determined as 30,015 Da by ESI-MS.

Variant N33Q (a conservative substitution) of SEQ ID NO: 1 will not be glycosylated even if expressed in fungal hosts. The non-glycosylated N33Q variant of SEQ ID NO: 1 showed similar efficacy as SEQ ID NO: 1 in an in vivo lipase screening test.

12. Enteric Coating of Granules Comprising Recombinantly Produced Purified Microbial Lipase

A coating solution is prepared by adding 1623.2 g of hydroxypropyl methylcellulose phthalate (HP 55), 90.2 g of triethyl citrate, 34.3 g of cetyl alcohol and 38.9 g of dimethicone 1000 to 14030 g of acetone at room temperature while stirring. 5025 g of granules (prepared analogously to the process as described in Example 2) are fed into a commercially available fluid bed coater and are spray-coated at a spray rate of 50-100 g/min and an air pressure of 1.5-2.5 bar with the coating solution as prepared above until the desired film-thickness of the coating is reached.

The product temperature of the lipase pellets is monitored with a suitable temperature sensor and maintained in the range between 37° C. and 49° C. during coating. The resulting lipase pellets are dried in a commercially available vacuum dryer (Vötsch type) at a temperature in a range between 35° C. and 50° C. for 12 hours.

13. Non-Functional Coating of Granules Comprising Recombinantly Produced Purified Microbial Lipase

500 g of granules (prepared analogously to the process as described in Example 2) are fed into a commercially available fluid bed coater and are spray-coated at a spray rate of 3-6 g/min and an air pressure of 0.8-1.2 bar with the coating solution as prepared above until the desired film-thickness of the coating is reached. A coating solution is prepared by adding 29.4 g of hydroxypropyl methylcellulose (HPMC E3 Premium LV) to 363.2 g of purified water at room temperature while stirring. The product temperature of the lipase pellets is monitored with a suitable temperature sensor and maintained in the range between 40° C. and 50° C. during coating.

14. Lipase Reference Standard (LRS)

(i). Manufacturing of the Lipase Reference Standard (LRS)

Solid lipase concentrate (20 g) obtained as described in Example 1 was used as starting material. The starting material was suspended in 180 mL demineralized water (pH adjusted to pH 6.0 with 20% acetic acid). 200 mL 10 mM succinic acid/NaOH solution and 2.0 M NaCl solution was added and the pH was adjusted to pH 6.0 to result in an almost clear solution. The mixture was then filtered through a 0.22 μm filtration unit. 20 mL of the filtrate was applied to a 20 mL acetylated decylamin-agarose (decyl-agarose) column (separation by Hydrophobic Interaction Chromatography, HIC) equilibrated in a solution of 10 mM succinic acid/NaOH, 2.0 M NaCl solution, pH 6.0. After a thorough wash of the column with the equilibration buffer, the column was stepwise eluted with 50 mM H₃BO₃/NaOH solution, pH 9.0, containing 30% isopropanol. The decyl-agarose step was repeated 19 times (20 times in total). All the eluates were combined (250 mL) and diluted to 15 L with demineralized water. The diluted lipase was applied to a 400 mL Q-sepharose FF column (separation by Ion Exchange Chromatography) equilibrated in 50 mM H₃BO₃/NaOH solution, pH 9.0. The column was washed thoroughly and the column was eluted with a linear NaCl gradient (0→0.5 M) over 3 column volumes. The eluted lipase peak (200 mL) was transferred to 20 mM HEPES/NaOH, 100 mM NaCl solution, 1 mM CaCl₂ solution, pH 7.0, by buffer exchange on a 1.4 L sephadex G25 column (separation by Size Exclusion Chromatography, SEC). The buffer exchanged lipase (300 mL) was filtered on a 0.22 μm filtration unit (=final product, 290 ml) and frozen in aliquots (5×50 ml, 1×30 ml, 1×10 ml).

The lipase solution obtained by this process was used as lipase reference standard (LRS).

(ii) Characterization

a) Identity

The identification of the lipase reference standard was confirmed by ESI-MS of the intact and deglycosylated protein and PMF (including ESI-MS/MS) with cleavage by Lysyl Endopeptidase (LysC) covering the typical variants of lipase with regards to N-terminal processing and glycosylation. Additionally, the disulfide-bridge connectivity was confirmed by protein digestion without reduction and reductive alkylation with identification of fragments by LC/MS.

b) Protein Purity

The protein purity of the lipase reference standard was determined as described in Example 6. The lipase reference standard has shown impurity of less than 0.1%.

c) Content

The content of the lipase reference standard was determined by amino acid analysis after hydrolysis using 6N HCl solution at 110° C. for 16 hrs under adequate vacuum. Separation was carried out by ion-exchange chromatography with post column derivatization (ninhydrine).

d) Specific Activity

The specific activity of the lipase reference standard was determined as described in Example 8.

15. Specific Activity of the Recombinantly Produced Purified Microbial Lipase as Compared with the Specific Activity of the Lipase Reference Standard

Solid lipase concentrate (20 g) obtained as described in Example 1 was used as starting material.

a) Manufacturing and Characterization of the Recombinantly Produced Purified Microbial Lipase.

The recombinantly produced purified microbial lipase was manufactured as described in Example 1. Determination of the lipase activity and of the specific activity of the recombinantly produced purified microbial lipase was performed as described in Example 7 and 8.

b) Manufacturing and Characterization of the Lipase Reference Standard

The lipase reference standard was manufactured and characterized as described in Example 14.

c) Comparison

The results for the purified lipase batches are shown in Table 2. The specific activity for the lipase reference standard (LRS) was 1,821,000 Units/g.

TABLE 2
Results for different recombinantly produced purified microbial lipase batches

| | Purified Lipase Batch# | | | | | | |
|---|---|---|---|---|---|---|---|
| | A | B | C | D | E | F | G |
| Lipase Activity | 1326409 | 1197396 | 1268180 | 1300219 | 1361471 | 1268226 | 1303934 |
| [U/g material] | | | | | | | |
| Protein Content | 79.7 | 80.6 | 79.1 | 80.6 | 83.1 | 80.7 | 83.9 |
| (Lipase)[%] | | | | | | | |
| Specific Activity | 1664252 | 1485603 | 1603262 | 1613175 | 1638353 | 1571532 | 1554153 |
| [U/g] | | | | | | | |
| % of LRS⁽¹⁾ | 91.39 | 81.58 | 88.04 | 88.59 | 89.97 | 86.30 | 85.35 |
| [%] | | | | | | | |

⁽¹⁾% LRS = 100* Specific Activity recombinantly produced purified microbial lipase/Specific Activity LRS

The specific activity of the recombinantly produced purified microbial lipase batches was at least 80% of the specific activity of the lipase reference standard.

All references, including publications, patent applications, and patents, cited herein are hereby incorporated by reference to the same extent as if each reference were individually and specifically indicated to be incorporated by reference and were set forth in its entirety herein.

The use of the terms “a,” “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

1-17. (canceled)

18. A pharmaceutical composition comprising:
a) granules containing:
i) a pharmaceutically acceptable core particle; and
ii) at least one coating layer coated on the core particle, said coating layer comprising at least one recombinantly produced purified microbial lipase;
wherein the recombinantly produced purified microbial lipase has a protein purity of at least 90 area-% (w/w) and a protein content of at least 60% (w/w).

19. The pharmaceutical composition of claim 18, wherein the specific activity of the purified microbial lipase is at least 80% of its maximum specific activity.

20. The pharmaceutical composition of claim 18, wherein the coating layer further comprises one or more enzyme stabilizing agents.

21. The pharmaceutical composition of claim 20, wherein the enzyme stabilizing agents are non-reducing carbohydrates.

22. The pharmaceutical composition of claim 21, wherein the non-reducing carbohydrates are selected from the group consisting of: sucrose, trehalose and maltitol.

23. The pharmaceutical composition of claims 18, wherein the coating layer further comprises one or more binding agents.

24. The pharmaceutical composition of claim 23, wherein the binding agents are selected from the group consisting of: hydroxypropylmethylcellulose, hydroxypropylcellulose, methylcellulose, carboxymethylcellulose, polyvinylpyrrolidon, dextrine and polyvinylalcohol.

25. The pharmaceutical composition of claim 18, wherein the purified microbial lipase is a lipase from Humicula lanuginosa.

26. The pharmaceutical composition of claim 18, further comprising pharmaceutical acceptable excipients.

27. The pharmaceutical composition of claim 26, wherein the composition is in a dosage form suitable for oral administration.

28. The pharmaceutical composition of claim 27, wherein the dosage form is selected from the group consisting of: capsules, granules, microtablets, pills, powders, sachets and tablets.

29. The pharmaceutical composition of claim 18, wherein the composition is used for the prevention or treatment of digestive disorders, pancreatic exocrine insufficiency, pancreatitis, cystic fibrosis, diabetes type I and/or diabetes type II.

30. A method of preventing or treating digestive disorders, pancreatic exocrine insufficiency, pancreatitis, cystic fibrosis, diabetes type I and/or diabetes type II by administering to a mammal in need thereof a therapeutically effective amount of the pharmaceutical composition of claim 18.

31. A method of preventing or treating digestive disorders, pancreatic exocrine insufficiency, pancreatitis, cystic fibrosis, diabetes type I and/or diabetes type II by administering to a mammal in need thereof a therapeutically effective amount of a recombinantly produced purified microbial lipase having a protein purity of at least 90 area-% and a protein content of at least 60% (w/w).

32. A method of manufacturing a pharmaceutical composition comprising the steps of:
a) providing pharmaceutically acceptable core particles;
b) providing a coating solution comprising at least one recombinantly produced purified microbial lipase having a purity of at least 90 area-% and a protein content of at least 60% (w/w);
c) coating one or more times the core particles of step a) with the coating solution of step b) to obtain granules containing at least one recombinantly produced purified microbial lipase; and
d) optionally, incorporating the granules of step c) into a suitable pharmaceutical dosage form.

33. A pharmaceutical composition prepared by the process comprising the steps of:
a) providing pharmaceutically acceptable core particles;
b) providing a coating solution comprising at least one recombinantly produced purified microbial lipase having a purity of at least 90 area-% and a protein content of at least 60% (w/w);
c) coating one or more times the core particles of step a) with the coating solution of step b) to obtain granules containing at least one recombinantly produced purified microbial lipase; and
d) optionally, incorporating the granules of step c) into a suitable pharmaceutical dosage form.
